(19) United States
(12) Patent Application Publication
(10) Pub. No.: US 2025/0360126 A1
(43) Pub. Date: Nov. 27, 2025

(54) TARGETING AN ENZYME REQUIRED FOR ACUTE MYELOID LEUKEMIA

(71) Applicant: Albert Einstein College of Medicine, Bronx, NY (US)

(72) Inventors: David M. Shechter, New Rochelle, NY (US); Kira Gritsman, Hastings on Hudson, NY (US)

(21) Appl. No.: 18/872,597

(22) PCT Filed: Jun. 6, 2023
(86) PCT No.: PCT/US2023/024566

§ 371 Date: Dec. 6, 2024

(51) Int. Cl. A61K 31/4985; A61K 31/4166; A61K 31/513; A61K 31/7105; A61K 31/713

(52) U.S. Cl. CPC A61K 31/4985; A61K 31/4166; A61K 31/513; A61K 31/7105; A61K 31/713

(57) ABSTRACT

Compositions and methods are provided for reducing NPM1 glutamate-glutamylation and for treating cancers characterized by overexpression of TTLL4 and/or expression of a mutant NPM1.

### CROSS-REFERENCE TO RELATED APPLICATIONS

This application claims the benefit of priority to U.S. Provisional Application No. 63/349,353 filed on Jun. 6, 2022, the contents of which are hereby incorporated by reference in their entirety.

### STATEMENT OF RIGHTS

This invention was made with government support under grant number W81XWH-21-1-0863 awarded by Department of Defense and GM135614 awarded by National Institutes of Health. The government has certain rights in the invention.

### BACKGROUND OF THE INVENTION

Mutations in the histone chaperone Nucleophosmin/NPM1 (annotated as NPM1c) are found in up to 35% of adult patients with acute myeloid leukemia (AML)(1). However, the mechanisms by which the NPM1c mutation transforms hematopoietic cells are still poorly understood. We recently identified a new and potentially targetable vulnerability in NPM1c AML cells: Tubulin-Tyrosine Ligase Like 4 (TTLL4)-catalyzed post-translational glutamate-glutamylation of the NPM1c protein. We previously showed that TTLL4-dependent glutamate-glutamylation of NPM2 (an embryonic NPM1 paralog) alters its chromatin assembly function (2, 3). Chromatin pathways are implicated in the initiation and progression of AML (4).

AML is a blood cancer that arises because of clonal expansion of malignant hematopoietic stem or progenitor cells. In AML patients, many frequently observed molecular events affect chromatin regulation (1, 5, 6). Chromatin—with a repeating nucleosomal unit of 147 bp of DNA wrapped around an octamer of histones H2A, H2B, H3, and H4—is the physiological form of the genome (7). H1 linker histones further compact chromatin (8) and generally repress gene expression (9-11). A key feature of NPM1c-mutant AML is increased expression of the HOXA and HOXB loci. HOX expression—important in organogenesis and body patterning—is both tightly coordinated with cell differentiation and frequently misregulated in leukemia (12). HOX gene downregulation occurs as cells progress to terminal differentiation. Aberrant expression of HOX genes in committed progenitors induces a leukemic state (12). In mouse models, expression of HOXA/B cluster genes is essential for the maintenance of NPM1c AML (13). One example chromatin mediated mechanism that leukemic cells use to maintain HOXA/B expression, including in NPM1c AML, is via increased activity of the H3K79 methyltransferase DOT1L (14-16). Increased DOT1L activity results in aberrant HOXA/B expression in committed progenitors, leading to a differentiation block.

The ability to target malignant hematopoietic stem or progenitor cells is an unmet need in AML treatment and is essential to reduce the risk of relapse. New therapies that target cancer stem cells are important for stopping cancer progression and recurrence. Identifying and developing new treatments for persistent cancer stem cells is critical for preventing relapse and progression.

### SUMMARY OF THE INVENTION

The present invention is based, at least in part, on the discovery that inhibition of TTLL4 activity can eliminate or reduce proliferation or induce differentiation of certain cells (e.g., cancerous cells). Without being bound by theory, this elimination or reduction in proliferation may be due to the reduction in glutamate-glutamylation of NPM1c that results from the inhibition of TTLL4.

One aspect of the present disclosure provides a method of reducing or eliminating cellular proliferation of a cell, the method comprising contacting the cell with a composition comprising an inhibitor of Tubulin-Tyrosine Ligase Like 4 (TTLL4), wherein the cell comprises an Nucleophosmin (NPM1) protein.

Another aspect of the present disclosure provides a method of reducing or eliminating glutamate-glutamylation of NPM1 in a cell, the method comprising contacting the cell with a composition comprising an inhibitor of TTLL4.

Numerous embodiments are further provided that can be applied to any aspect of the present invention and/or combined with any other embodiment described herein. For example, in one embodiment, the inhibitor of TTLL4 is an inhibitory nucleic acid, a small molecule inhibitor, or an antibody, or fragment thereof, that specifically binds TTLL4 or a nucleic acid molecule encoding TTLL4. In some embodiments, the inhibitor is a small molecule, which may specifically bind to TTLL4. As described herein, the inhibitor (e.g., the small molecule) may interact with one or more of amino acid residues F666, 1719, K721, R727, G728, Q749, R750, Y751, L752, K762, D764, R766, R788, H807, L808, T809, N810, Y811, S812, K815, K833, D893, E906, M895, L905, E906, N908, 1909, S912, H914, D920, and K924 of TTLL4. In some embodiments, the inhibitory nucleic acid molecule is an siRNA, miRNA, or shRNA. The inhibitory nucleic acid molecule can be at least at least 80%, 85%, 90%, 95%, or 100% complementary to the nucleic acid sequence encoding the TTLL4. In some embodiments, the inhibitory nucleic acid molecule comprises at least one modified nucleotide.

In some embodiments, the composition that contacts the cell further comprises a vector comprising a nucleic acid sequence encoding the inhibitory nucleic acid molecule. The vector can be an expression vector. In some embodiments, the vector is a viral vector.

In some embodiments, the methods further comprise detecting the glutamate-glutamylation levels of NPMINPM1c prior to contacting the cell, after contacting the cell, or both prior and after contacting the cell.

In another aspect a method is provided for reducing or eliminating cellular proliferation of a cell, the method comprising contacting the cell with a guide RNA (gRNA) that is at least 80%, 85%, 90%, 95%, or 100% complementary to a nucleic acid sequence in the TTLL4 gene and one or more components of a CRISPR/Cas system or a nucleic acid molecule encoding the one or more components of a CRISPR/Cas system, wherein the cell comprises NPM1.

Another aspect provides a method of reducing or eliminating glutamate-glutamylation of NPM1 in a cell, the method comprising contacting the cell with a guide RNA (gRNA) that is at least 80%, 85%, 90%, 95%, or 100% complementary to a nucleic acid sequence in the TTLL4 gene and one or more components of a CRISPR/Cas system or a nucleic acid molecule encoding the one or more components of a CRISPR/Cas system.

Yet another aspect provides a method of modifying the TTLL4 gene in a cell, the method comprising contacting the cell with a guide RNA (gRNA) that is at least 80%, 85%, 90%, 95%, or 100% complementary to a nucleic acid sequence in the TTLL4 gene and one or more components of a CRISPR/Cas system or a nucleic acid molecule encoding the one or more components of a CRISPR/Cas system, wherein the cell comprises NPM1.

Numerous embodiments are further provided that can be applied to any aspect of the present invention and/or combined with any other embodiment described herein. For example, in one embodiment, the gRNA comprises at least one modified nucleotide. In some embodiments, the nuclease is a Cas9 nuclease. The Cas9 nuclease can be a Cas9 nickase or a Cas9 cleavase. In some embodiments, the Cas9 nuclease introduces a double-stranded break in the TTLL4 gene, thereby reducing or silencing expression of the TTLL4 gene.

In some embodiments, the cell is a hematopoietic/progenitor stem cell. In some embodiments, the cell is an acute myeloid leukemic cell. In some embodiments, the contacting of the cell is in vitro or in vivo.

Another aspect of the present disclosure is a cell made by any of the methods described herein.

In yet another aspect, a method is provided for treating a cancer in a subject, the method comprising administering to the subject a composition comprising an inhibitor of TTLL4. Numerous embodiments are further provided that can be applied to any aspect of the present invention and/or combined with any other embodiment described herein. For example, in some embodiments, the inhibitor of TTLL4 is an inhibitory nucleic acid, a small molecule inhibitor, or an antibody, or fragment thereof, that specifically binds TTLL4 or a nucleic acid molecule encoding TTLL4. In some embodiments, the inhibitor is a small molecule. The small molecule, in some embodiments, can specifically bind to TTLL4. In some embodiments, the small molecule interacts with amino acid residue F666, 1719, K721, R727, G728, Q749, R750, Y751, L752, K762, D764, R766, R788, H807, L808, T809, N810, Y811, S812, K815, K833, D893, E906, M895, L905, E906, N908, 1909, S912, H914, D920, and/or K924 of TTLL4.

In some embodiments, the inhibitory nucleic acid molecule is an siRNA, miRNA, or shRNA. In some embodiments, the inhibitory nucleic acid molecule is at least at least 80%, 85%, 90%, 95%, or 100% complementary to the nucleic acid sequence encoding the TTLL4. In some embodiments, the inhibitory nucleic acid molecule comprises at least one modified nucleotide.

The composition administered to the subject may further comprise a vector comprising a nucleic acid sequence encoding the inhibitory nucleic acid molecule. In some embodiments, the vector is an expression vector. In some embodiments, the vector is a viral vector.

In some embodiments, the method of treating a cancer further comprises detecting the glutamate-glutamylation levels of NPM1 prior to contacting the cell, after contacting the cell, or both prior and after contacting the cell.

Another aspect of this disclosure provides a method of treating a cancer in a subject, the method comprising administering to the subject a guide RNA (gRNA) that is at least 80%, 85%, 90%, 95%, or 100% complementary to a nucleic acid sequence in the TTLL4 gene and one or more components of a CRISPR/Cas system or a nucleic acid molecule encoding the one or more components of a CRISPR/Cas system. In some embodiments, the gRNA comprises at least one modified nucleotide. Exemplary nucleotide modifications include, but are not limited to, 2′-deoxy, 2′-fluoro (2′-F), 2′-O-methyl (M), 2′-O-methyl 3′phosphorothioate (MS), 2′-O-methyl 3′ thioPACE (MSP), phosphorothioate (PS), LNA-locked nucleic acid, replacement of a ribonucleotide with a deoxyribonucleotide, MP, 2′F-ANA, 2′F-4′-Cα-Ome, 2′, 4′-diCα-Ome, BNA^{NC} (N-Me), S-constrained ethyl (CET), unlocked nucleic acid (UNA), 2′5′-RNA, and butane. Inclusion of modified nucleotides has been discussed in Sakovina et al. (2022) Int. J. Mol. Sci. 23:13460; Allen et al. (2021) Frontiers in Genome Editing 2:617910; and Rozners (2022) J. Am. Chem. Soc. 144 (28): 12584-12594, each of which is incorporated herein by reference. The nuclease may be a Cas9 nuclease, such as a Cas9 nickase or a Cas9 cleavase. The Cas9 nuclease may introduce a double-stranded break in the TTLL4 gene in a cell, thereby reducing or silencing expression of the TTLL4 gene in the cell. In some embodiments the cell is a hematopoietic/progenitor stem cell. In some embodiments, the cell is an acute myeloid leukemic cell. In some embodiments, the gRNA and the CRISPR/Cas system are coadministered. In some embodiments, the gRNA and the CRISPR/Cas system are present in a pharmaceutical composition further comprising a pharmaceutically acceptable carrier. The gRNA may be present in a first pharmaceutical composition further comprising a pharmaceutically acceptable carrier and the CRISPR/Cas system is present in a second pharmaceutical composition further comprising a pharmaceutically acceptable carrier. In some embodiments, the gRNA and the CRISPR/Cas system are administered sequentially.

In some embodiments of the methods of treating a cancer further comprise detecting the level of TTLL4 protein or polynucleotide and/or NPM1 glutamate-glutamylation. In some the detecting is performed prior to administration or after administration. In some embodiments, the detecting is performed prior to and after administration, wherein a decrease in the level of TTLL4 and/or NPM1 glutamate-glutamylation is indicative of therapeutic effectiveness.

In any of the methods presented herein, the NPM1 is in certain embodiments NPM1c.

### BRIEF DESCRIPTION OF THE DRAWINGS

FIGS. 1A and 1B show the domain structure of NPM1 and mutant NPM1c proteins. FIG. 1A is a schematic showing the hydrophobic core domain (blue box) with disordered C-terminal tail (black line). A1, A2, A3=Acidic Patches; NES=Nuclear Export Signal; NLS=Nuclear Localization Signal. The three-helical bundle (3HB) binds RNA and promotes nucleolar localization. The indicated histone binding site is also the site of glutamate-glutamylation (sequence of likely glutamate-glutamylation sites is indicated). The NPM1c (bottom) somatic mutation disrupts the folding of the 3HB (x). FIG. 1B shows the pentameric organization of the wild type (top) and mutant (bottom).

FIGS. 2A-D shows that TTLL4 catalyzes NPM glutamate-glutamylation, CCP5 catalyzes deglutamate-glutamylation, and NPM1c is preferentially glutamylated. FIG. 2A show that TTLL4 adds a glutamic acid to a protein, while CCP5 is responsible for deglutamylating. FIG. 2B shows that TTLL4 glutamylates full-length NPM1, but not NPM1 with a truncated C-terminal intrinsically disordered region (IDR). FIG. 2C is a blot showing that the CCP5 enzyme deglutamylates NPM2. FIG. 2D is a Western blot showing that TTLL4 overexpression increases glutamate-glutamylation of NPM1c relative to wild type NPM1.

FIGS. 3A and 3B illustrate that TTLL4 catalyzes NPM1 and NPM1c glutamate-glutamylation. FIG. 3A is a Western blot of an AML3-NPM1c-degron cell line with a doxycycline (DOX)-inducible TTLL4 shRNA knockdown. Scrambled shRNA or TTLL4 shRNA cells were treated +/−DOX, resulting in loss of glutamate-glutamylation on both NPM1c-degron and NPM1 wild type proteins (top gel, +DOX). FIG. 3B is a summary of a qRT-PCR analysis of shTTLL4 cells treated with DOX.

FIGS. 4A-4D show the results of assays characterizing TTLL4 function in vitro. FIG. 4A shows Cell Titer Glo assays of OCI-AML3 cells transduced with TTLL4 or scrambled shRNAs. FIG. 4B shows phenotypic assays of Cas9-OCI-AML3 cells ±TTLL4 deletion. Day 0: RFP+ sort for of sgRNA-expressing cells resulting in significantly reduced colony formation. Shown-Total colonies (CFU) at day 7. FIG. 4C shows results from a flow cytometry apoptosis analysis at day 8. Annexin V+7-AAD-population represents apoptotic cells. Error bars indicate mean±SEM. **p≤0.01; ***p≤0.001. NT-sgRNA: non-targeting control; sgRNA-1/2/3/4: TTLL4 deletion. FIG. 4D is a set of images of Wright-Giemsa staining of cytospins of OCI-AML3 cells with TTLL4 kd.

FIGS. 5A and 5B illustrate a transcriptome analysis of TTLL4 and NPM1c loss. FIG. 5A shows the results of a principal component analysis (PCA) of RNA-Seq. Biological triplicates are represented; each circled and annotated. FIG. 5B shows a gene set enrichment analysis of each condition, relative to the control untreated OCI-AML3 cells. Normalized enrichment scores (NES)(FDR<0.05) from the analysis of each of the listed gene sets are shown in a heatmap.

FIG. 6 is a graph showing the survival rates for all TCGA cancers. The data show that elevated TTLL4 expression leads to significantly and dramatically poorer overall survival in all human cancers.

FIGS. 7A-7C show the TTLL4 catalytic mechanism and substrates. FIG. 7A is a putative reaction mechanism: protein glutamic acid proceeds through ATP-dependent phosphorylation of the gamma-carboxyl followed by glutamate acyl substitution, forming an isopeptide bond with a resultant gamma-glutamate post-translational modification. FIG. 7B is a Western blot showing kinase reaction showing TTLL4 dependent 32P-labeled phosphorylation of Nap1 (a TTLL4 substrate protein). Subsequent addition of glutamate removes the phosphorylation. FIG. 7C is a quantification of the phosphorylation detected in FIG. 7B revealing 1st order kinetics.

FIGS. 8A-8E shows the basis for virtual screening: TTLL4 model, pharmacophores, and ligands. FIG. 8A shows an alphafold-multimer docked model of TTLL4 catalytic domain with acidic IDR substrate (orange cartoon). TTLL4 is colored by electrostatic potential (blue =basic; red=acidic). FIG. 8B shows the hydrophobic surfaces of TTLL4 (yellow), revealing the ATP, glu, and peptide binding pockets. The hydrophobic anchor (F666, 1909) are unique to TTLL4, providing an opportunity for specific pharmacophore targeting. FIG. 8C is a cartoon representation of catalytic site, revealing ATP and initiation analog (site of glutamate-glutamylation). FIG. 8D shows a structure-guided virtual screening with pharmacophore residues indicated in gray (R727, R788, N810, K833, D893, E906, N908, F666, 1909). FIG. 8E shows a ligand-guided virtual screening: initiation intermediate (chemical structure, left; conformation of molecule, right).

FIGS. 9A-9C show Homo sapiens TTLL4 catalytic domain enzymatic parameters. FIG. 9A shows that TTLL4 exhibited Michaelis-Menten kinetics with a Km ˜257 μM and kcat of 12.5/min with saturating protein and ATP substrates. FIG. 9B shows that for ATP, TTLL4 had a Km ˜41 μM and kcat of 12.7/min. FIG. 9C shows that TTLL4 had optimal activity near pH 9.0.

FIG. 10 shows that NPM1c is a better TTLL4 substrate than is wild type NPM1. Immunoblot demonstrating in vitro glutamate-glutamylation of purified recombinant NPM1 and NPM1c proteins at multiple time points upon addition of recombinant TTLL4 enzyme. DB71=membrane stain/loading control.

FIG. 11 shows a candidate small molecule inhibitor having anti-TTLL4 activity.

FIG. 12 is a scatter plot showing results from a screening assay of small molecule inhibitors. TTLL4 activity was measured by immuno-detection of the glutamylated protein substrate. Small molecules were screened in duplicate at 50 μM. Scatter plot of an example 100 of approximately 400 screened compounds are shown. Compounds with demonstrated potency in duplicate (further screened compounds) were subsequently titrated to determine an IC50 (not shown).

FIG. 13 Is a plot showing theTTLL4 activity profile of titration of candidate inhibitor 1.

FIG. 14A-B Raw enzyme assay data for candidate inhibitor 3 (14A). Chemical structure of Candidate inhibitor 3, with an in vitro TTLL4 IC50 of 19.7 μM (14B).

FIGS. 15A-D are schematics showing potential docking of the different diasteromeric forms of candidate inhibitor 2. FIG. 15A shows the (S,S) form, FIG. 15B shows the (S,R) form, FIG. 15C shows the (R,S,) form, and FIG. 15D shows the (R,R) form.

FIG. 16 is a schematic potential docking of candidate inhibitor 1.

### DETAILED DESCRIPTION OF THE INVENTION

It has been determined herein that the inhibition of TTLL4 reduces proliferation of cancerous cells from a subject having acute myeloid leukemia (AML). AML is a blood cancer that arises because of clonal expansion of malignant hematopoietic stem or progenitor cells. In AML patients, many frequently observed molecular events affect chromatin regulation (1, 5, 6). Chromatin—with a repeating nucleosomal unit of 147 bp of DNA wrapped around an octamer of histones H2A, H2B, H3, and H4—is the physiological form of the genome (7). H1 linker histones further compact chromatin (8) and generally represses gene expression (9-11). A key feature of NPM1c-mutant AML is increased expression of the HOXA and HOXB loci. HOX expression-important in organogenesis and body patterning—is both tightly coordinated with cell differentiation and frequently misregulated in leukemia (12). HOX gene downregulation occurs as cells progress to terminal differentiation. Aberrant expression of HOX genes in committed progenitors induces a leukemic state (12). In mouse models, expression of HOXA/B cluster genes is essential for the maintenance of NPM1c AML (13). One example of a chromatin-mediated mechanism that leukemic cells use to maintain HOXA/B expression, including in NPM1c AML, is increased activity of the H3K79 methyltransferase, DOT1L (14-16). Increased DOT1L activity results in aberrant HOXA/B expression in committed progenitors, which can block differentiation.

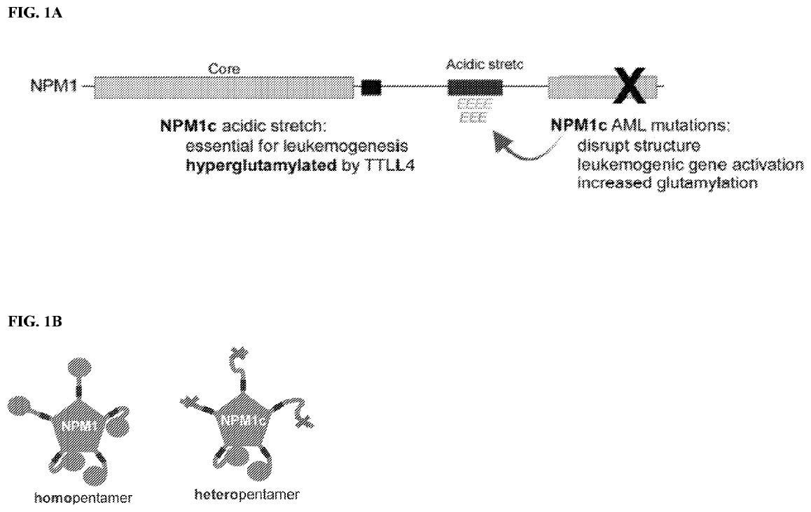

NPM1 is a ubiquitously expressed pentameric histone chaperone-binding core and linker histones—with two major domains: an N-terminal core oligomerization domain and an intrinsically disordered C-terminal tail ending in a three-helix bundle (3HB)(FIGS. 1A and 1B)(17). The acidic stretches in the C-terminal intrinsically disordered region (IDR) are responsible for histone binding (18, 19). Histone chaperones, like NPM1, regulate chromatin by (20): 1) preventing histone aggregation; 2) facilitating cytoplasmic-nuclear transport of histones; and 3) promoting either histone deposition or histone removal from DNA. In the NPM1 paralog NPM2, hindering access of histones to the acidic stretches has shown that the C-terminal tail has an autoregulatory role in both histone binding and deposition (21). NPM1c AML mutations occur in the C-terminal 3HB (5). In addition to potential modulation of histone chaperone function, these mutations are correlated with NPM1c's aberrant cytoplasmic localization (19, 22-24).

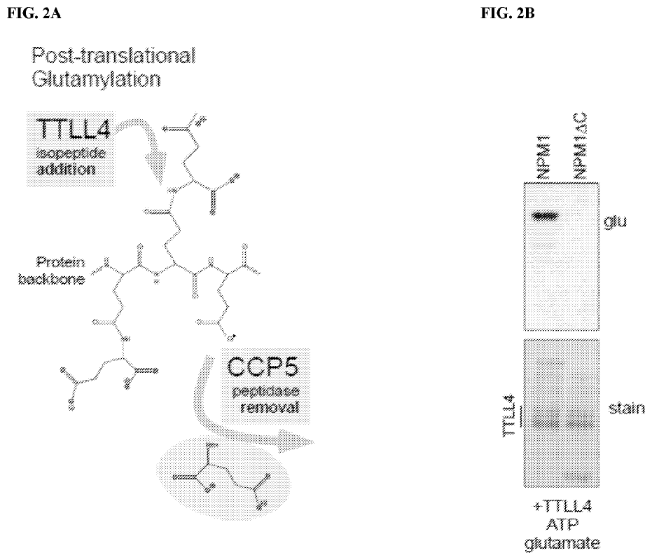

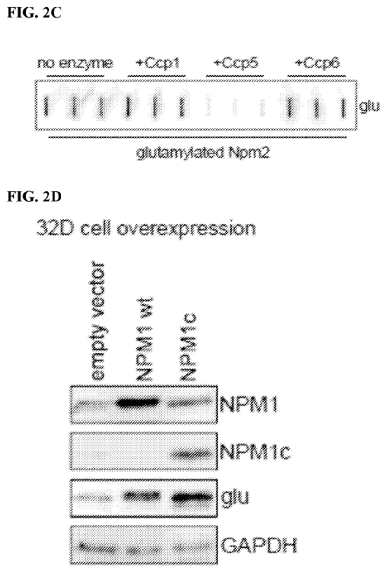

Post-translational glutamate-glutamylation is found on histone chaperones, including NPM1 (FIG. 2a)(3, 25-30). NPM1 is glutamylated by TTLL4 and glutamate-glutamylation is removed by CCP5 (cytosolic carboxypeptidase-like 5)(FIGS. 2B-2D). TTLL4 and CCP5 both have oncogenic roles (27, 29, 31-34). Consistent with a chromatin regulatory mechanism, NPM2 glutamate-glutamylation enhances its affinity for histones (2).

### I. Definitions

The articles “a” and “an” are used herein to refer to one or to more than one (i.e. to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “altered amount” or “altered level” refers to increased or decreased expression or activity level in a subject sample or cell, as compared to the expression or activity level of a biomarker nucleic acid or protein in a control sample or cell. Furthermore, an altered amount of a protein may be determined by detecting posttranslational modifications such as glutamate-glutamylation status of the marker, which may affect the expression or activity of the protein.

The amount of a nucleic acid or protein in a subject is “significantly” higher or lower than the normal amount of the nucleic acid or protein, if the amount of the nucleic acid or protein is greater or less, respectively, than the normal level by an amount greater than the standard error of the assay employed to assess amount, and preferably at least 10%, 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the amount of the nucleic acid or protein in the subject can be considered “significantly” higher or lower than the normal amount if the amount is at least about two, and preferably at least about three, four, or five times, higher or lower, respectively, than the normal amount of the nucleic acid or protein.

The term “altered level of expression” of a nucleic acid or protein refers to an expression level or copy number of the nucleic acid or protein in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the nucleic acid or protein in a control sample (e.g., sample from a healthy subjects not having the associated disease) and preferably, the average expression level or copy number of the nucleic acid or protein in several control samples. The altered level of expression is greater or less than the standard error of the assay employed to assess expression or copy number, and is preferably at least twice, and more preferably three, four, five or ten or more times the expression level or copy number of the nucleic acid or protein in a control sample (e.g., sample from a healthy subjects not having the associated disease) and preferably, the average expression level or copy number of the biomarker in several control samples.

The term “altered activity” of a protein refers to an activity of the protein which is increased or decreased in a disease state, e.g., in a cancer sample, as compared to the activity of the protein in a normal, control sample. Altered activity of the protein may be the result of, for example, altered expression of the protein, altered protein level of the biomarker, altered structure of the protein, or, e.g., an altered interaction with other proteins involved in the same or different pathway as the protein of interest or altered interaction with transcriptional activators or inhibitors.

The term “altered structure” of a nucleic acid or protein includes the presence of mutations or allelic variants within the nucleic acid or protein, e.g., mutations which affect expression or activity of the nucleic acid or protein, as compared to the normal or wildtype gene or protein. For example, mutations include, but are not limited to substitutions, deletions, or addition mutations. Mutations may be present in the coding or non-coding region of the nucleic acid.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multispecific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16:778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′) 2 fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies encompassed by the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies encompassed by the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluid that are normally not (e.g. amniotic fluid, aqueous humor, bile, blood and blood plasma, cerebrospinal fluid, cerumen and earwax, Cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, and vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of oncogenes, such as c-MYC. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic or myeloid leukemia (AML, myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In some embodiments, the cancer is AML. In other embodiments, the cancer is renal cell kidney cancer or melanoma. In some embodiments, the cancer is any in which TTLL4 is significantly overexpressed compared to normal cells of the same originating tissue.

The term “coding region” refers to regions of a nucleotide sequence comprising codons which are translated into amino acid residues, whereas the term “non-coding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “conjoint therapy” and “combination therapy,” as used herein, refer to the administration of two or more therapeutic substances, e.g., combinations of agents that target different biomarkers, multiple agents that target the same biomarker, combination of anti-biomarker agents and additional anti-cancer agents like chemotherapy, and the like, and combinations thereof. The different agents comprising the combination therapy can be administered concomitant with, prior to, or following the administration of one or more therapeutic agents.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods encompassed by the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods encompassed by the present invention are not limited to use of a specific cut-point in comparing the level of expression product in the test sample to the control.

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is determining whether to provide targeted therapy against a cancer to provide anti-cancer therapy e.g., therapy with at least one agent that inhibits TTLL4 or NPM1c glutamate-glutamylation. Another example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “inhibitor” refers to a compound having the ability to inhibit a biological function of a target biomolecule, for example, an mRNA or a protein, whether by decreasing the activity or expression of the target biomolecule. Accordingly, the term “inhibitor” is defined in the context of the biological role of the target biomolecule.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction,” when referring to an interaction between two molecules, refers to the physical contact (e.g., binding) of the molecules with one another. Generally, such an interaction results in an activity (which produces a biological effect) of one or both of said molecules.

A “kit” is any manufacture (e.g. a package or container) comprising at least one reagent, e.g. a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The term “neoadjuvant therapy” refers to a treatment given before the primary treatment. Examples of neoadjuvant therapy can include chemotherapy, radiation therapy, and hormone therapy. For example, in treating breast cancer, neoadjuvant therapy can allows patients with large breast cancer to undergo breast-conserving surgery.

The “normal” level of expression of a biomarker nucleic acid, or protein is the activity/level of expression in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer.

The “normal” level of expression and/or activity of a biomarker (e.g., TTLL4, NPM1c) is the level of expression and/or activity of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples. The same determination can be made to determine overactivity or underactivity. Such “significance” levels can also be applied to any other measured parameter described herein, such as for expression, inhibition, cytotoxicity, cell growth, and the like.

“Pharmacophore,” as used herein is a 3D arrangement of a functional molecule (e.g., TTLL4) that is crucial to attach or bind to an active site of an enzyme or molecule.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to anti-cancer therapy, such as therapy with at least one agent that inhibits at TTLL4. Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC) and/or biomarker target, or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to a particular anti-cancer therapy (e.g., therapy with at least one agent that inhibits TTLL4) or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., AML, solid tumors, such as, melanoma, and renal cell kidney cancer), development of one or more clinical factors, development of intestinal cancer, or recovery from the disease.

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene encompassed by the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn, G. and Cullen, B. (2002) J. of Virology 76 (18): 9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs, shRNAs, or other RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present invention further comprises obtaining the sample from the individual prior to detecting or determining the presence or level of at least one marker in the sample.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA Apr.; 9 (4): 493-501 incorporated by reference herein).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides)(Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl goups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene-(CH₂)—, ethylene-(CH₂CH₂)—, n-propylene-(CH₂CH₂CH₂)—, isopropylene-(CH₂CH (CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to a cycloalkyl group as hereinbefore defined substituted with at least one halogen.

“Heterocycloalkyl” refers to an cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” or “heterocycloalkyl” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

The term “subject” refers to any healthy animal, mammal or human, or any animal, mammal or human afflicted with a cancer, e.g., lung, ovarian, pancreatic, liver, breast, prostate, and colon carcinomas, as well as melanoma and multiple myeloma. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods encompassed by the present invention may be administered in a sufficient amount to produce a reasonable benefit/risk ratio applicable to such treatment.

The terms “therapeutically-effective amount” and “effective amount” as used herein means that amount of a compound, material, or composition comprising a compound encompassed by the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC50 (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

The term “TTLL4” refers to tubulin tyrosine ligase like 4, a polyglutamylase. In addition to glutamate-glutamylation of tubulin proteins, TTLL4 protein can promote glutamate-glutamylation of histone chaperones and other proteins involved in the chromatin structure. For example, TTLL4 has been correlated with polyglutamate-glutamylation levels NPM1c, which is a variant of NPM1 and is associated with acute myeloid leukemia (AML).

>NP_055455.3 tubulin monoglutamylase TTLL4 [Homo sapiens]
(SEQ ID NO: 1)
MASAGTQHYSIGLRQKNSFKQSGPSGTVPATPPEKPSEGRVWPQAHQQVKPIWKLEKKQVETLSAGLGPG
LLGVPPQPAYFFCPSTLCSSGTTAVIAGHSSSCYLHSLPDLFNSTLLYRRSSYRQKPYQQLESFCLRSSP
SEKSPFSLPQKSLPVSLTANKATSSMVFSMAQPMASSSTEPYLCLAAAGENPSGKSLASAISGKIPSPLS
SSYKPMLNNNSFMWPNSTPVPLLQTTQGLKPVSPPKIQPVSWHHSGGTGDCAPQPVDHKVPKSIGTVPAD
ASAHIALSTASSHDTSTTSVASSWYNRNNLAMRAEPLSCALDDSSDSQDPTKEIRFTEAVRKLTARGFEK
MPRQGCQLEQSSFLNPSFQWNVLNRSRRWKPPAVNQQFPQEDAGSVRRVLPGASDTLGLDNTVFCTKRIS
IHLLASHASGLNHNPACESVIDSSAFGEGKAPGPPFPQTLGIANVATRLSSIQLGQSEKERPEEARELDS
SDRDISSATDLQPDQAETEDTEEELVDGLEDCCSRDENEEEEGDSECSSLSAVSPSESVAMISRSCMEIL
TKPLSNHEKVVRPALIYSLFPNVPPTIYFGTRDERVEKLPWEQRKLLRWKMSTVTPNIVKQTIGRSHFKI
SKRNDDWLGCWGHHMKSPSFRSIREHQKLNHFPGSFQIGRKDRLWRNLSRMQSRFGKKEFSFFPQSFILP
QDAKLLRKAWESSSRQKWIVKPPASARGIGIQVIHKWSQLPKRRPLLVQRYLHKPYLISGSKFDLRIYVY
VTSYDPLRIYLFSDGLVRFASCKYSPSMKSLGNKFMHLTNYSVNKKNAEYQANADEMACQGHKWALKALW
NYLSQKGVNSDAIWEKIKDVVVKTIISSEPYVTSLLKMYVRRPYSCHELFGFDIMLDENLKPWVLEVNIS
PSLHSSSPLDISIKGQMIRDLLNLAGFVLPNAEDIISSPSSCSSSTTSLPTSPGDKCRMAPEHVTAQKMK
KAYYLTQKIPDQDFYASVLDVLTPDDVRILVEMEDEFSRRGQFERIFPSHISSRYLRFFEQPRYFNILTT
QWEQKYHGNKLKGVDLLRSWCYKGFHMGVVSDSAPVWSLPTSLLTISKDDVILNAFSKSETSKLGKQSSC
EVSLLLSEDGTTPKSKKTQAGLSPYPQKPSSSKDSEDTSKEPSLSTQTLPVIKCSGQTSRLSASSTFQSI
SDSLLAVSP
>NM_014640.5 Homo sapiens tubulin tyrosine ligase like 4 (TTLL4), mRNA
(SEQ ID NO: 2)
CTCTTCTTCCAGACTCTCGGTCTGTCCGCTGGGGGCGCGCGCGGTGTGTGGCAGGCGGCAGCGGCGCTGG
CGGCCGAGTGCGCTTGTCACGCGTGGCGGTGCGTGGTTGCTAGGGGCGCCTGAGGCTGCCGGGTAGCCCA
GCAGGCCGAGGGAGGAAGTAGCGTGGAGCCGGTGCCGAGCCGGGGCGAAGCTGGATCCCCTAGATAGACT
GTCTTCAAGCTCACTGATATTTTCCTCTGCTTGATCCATTGTGCTGTTGAGAGCCTCTAGTAAATTTTTC
AGACTGACAGACTTCAAGGATGCAGCTGCTACTACCGGAGGTGTGTGGCACCTTACCTCAGCAAGGCCAT
GAGACCGTGTGGCCATGATGTGGGCCCCTCATGGCCTCAGCAGGAACACAGCACTATAGTATTGGCCTCC
GCCAGAAAAACAGCTTCAAGCAGAGTGGTCCCTCAGGCACAGTACCTGCCACGCCACCTGAGAAACCCTC
GGAGGGCAGAGTCTGGCCTCAGGCCCATCAGCAAGTGAAGCCAATCTGGAAGCTGGAAAAGAAGCAAGTG
GAGACACTGTCAGCAGGGTTGGGCCCAGGCCTCTTGGGCGTCCCACCCCAGCCAGCATATTTCTTTTGCC
CCAGCACTTTATGTAGCTCTGGGACCACGGCTGTCATTGCAGGCCACAGCAGTTCCTGTTACCTACACTC
TCTCCCGGACTTGTTCAACAGCACCCTGCTATACCGCCGCTCCAGCTATAGGCAAAAACCGTACCAGCAA
CTGGAGTCTTTCTGCTTGCGTTCGAGCCCGTCAGAAAAAAGCCCTTTTTCTCTCCCTCAAAAGAGCCTCC
CTGTCAGTCTCACTGCCAACAAGGCCACTTCTTCCATGGTCTTCTCCATGGCCCAGCCCATGGCCTCCTC
ATCCACAGAACCATACCTCTGCTTGGCAGCGGCTGGGGAAAACCCTTCAGGGAAGAGCCTGGCCTCTGCC
ATCTCAGGGAAGATCCCATCTCCACTCTCTTCCTCCTATAAGCCCATGCTGAATAATAATTCCTTCATGT
GGCCAAATAGCACGCCAGTGCCTTTATTGCAGACCACACAGGGCCTGAAGCCAGTATCGCCACCCAAGAT
CCAGCCTGTCTCCTGGCATCATTCAGGGGGTACTGGAGACTGTGCACCGCAGCCTGTTGACCATAAGGTG
CCCAAAAGCATTGGCACTGTCCCAGCTGATGCCAGTGCCCATATCGCCTTGTCTACCGCTAGCTCCCACG
ACACATCCACCACCAGTGTTGCCTCTTCCTGGTATAACCGGAATAACTTAGCCATGAGGGCAGAGCCACT
TTCCTGTGCTCTGGATGACAGCTCTGATTCCCAGGATCCAACTAAGGAGATTCGGTTCACTGAGGCCGTG
AGGAAATTGACCGCAAGAGGCTTTGAGAAGATGCCGAGGCAAGGCTGCCAGCTTGAACAGTCTAGTTTCC
TGAACCCCAGCTTCCAGTGGAATGTCCTCAACAGGAGCAGGCGGTGGAAACCTCCTGCGGTAAATCAGCA
GTTTCCTCAGGAGGATGCTGGATCGGTCAGGCGGGTCCTCCCTGGTGCCTCAGATACCTTGGGGTTGGAC
AATACAGTCTTCTGTACCAAGCGTATCAGCATTCACCTCCTTGCCTCACATGCCAGTGGGCTCAATCACA
ACCCTGCCTGTGAATCTGTAATTGACTCCTCAGCATTTGGAGAAGGCAAAGCTCCAGGTCCCCCTTTTCC
TCAAACTCTTGGCATAGCCAACGTGGCCACCCGCCTCTCTTCCATCCAGCTGGGCCAGTCTGAGAAGGAG
AGACCTGAGGAGGCCAGGGAGCTGGACTCATCTGATAGGGATATTAGTTCAGCTACTGACCTCCAGCCAG
ATCAGGCTGAGACTGAAGATACAGAAGAAGAACTAGTAGATGGTTTGGAAGACTGTTGTAGCCGTGATGA
GAATGAAGAGGAGGAGGGAGACTCAGAGTGCTCCTCATTAAGTGCTGTCTCCCCCAGCGAATCGGTGGCC
ATGATCTCTAGAAGCTGTATGGAAATTCTGACCAAACCCCTTTCCAATCATGAGAAAGTTGTCCGACCAG
CCCTCATCTACAGTCTCTTTCCCAACGTTCCCCCTACCATCTATTTTGGCACTCGGGATGAGAGAGTGGA
GAAACTTCCCTGGGAACAGAGGAAGTTGCTCCGATGGAAGATGAGCACAGTGACCCCCAACATTGTCAAG
CAGACCATTGGACGGTCCCACTTCAAAATCAGCAAAAGAAACGATGACTGGCTGGGCTGCTGGGGTCACC
ACATGAAGTCTCCTAGTTTCCGATCCATTCGAGAGCATCAGAAGCTAAACCATTTCCCAGGCTCATTCCA
GATTGGGAGGAAGGACCGGCTATGGCGGAACCTGTCACGTATGCAGAGCCGCTTTGGCAAGAAGGAGTTC
AGTTTCTTCCCCCAGTCCTTTATCCTGCCCCAGGACGCCAAGCTCCTGCGCAAAGCGTGGGAGAGCAGCA
GCCGCCAAAAGTGGATTGTGAAGCCACCAGCATCAGCTCGAGGCATTGGCATCCAGGTTATTCACAAGTG
GAGTCAGCTCCCCAAGCGAAGGCCCCTCCTGGTACAGAGGTATCTACACAAACCCTACCTCATCAGCGGC
AGCAAGTTTGACCTGCGGATCTATGTTTATGTCACTTCCTACGATCCTCTGCGGATTTACCTCTTTTCAG
ATGGACTGGTCCGCTTTGCCAGTTGCAAGTATTCGCCTTCCATGAAGAGCCTTGGCAATAAGTTCATGCA
CCTGACCAACTACAGTGTCAATAAAAAGAATGCCGAGTACCAGGCCAATGCAGATGAAATGGCTTGCCAG
GGCCACAAATGGGCACTGAAGGCTTTGTGGAACTACCTGAGCCAGAAGGGAGTCAATAGCGACGCCATCT
GGGAGAAGATAAAGGATGTTGTTGTCAAAACTATCATCTCGTCAGAGCCCTATGTGACCAGCCTGCTCAA
GATGTATGTGCGACGGCCCTATAGCTGCCATGAACTCTTTGGTTTTGACATCATGCTAGACGAAAACCTC
AAGCCCTGGGTCCTGGAAGTCAACATTTCCCCAAGCCTCCACTCCAGCTCTCCACTGGATATCAGCATCA
AAGGCCAGATGATTCGTGACCTTCTGAATCTGGCAGGTTTTGTCCTGCCCAATGCAGAGGATATCATTTC
CAGCCCCAGCAGCTGCAGCAGCTCCACCACCAGCCTGCCCACCTCCCCTGGGGACAAATGTCGAATGGCT
CCAGAGCATGTCACTGCACAGAAGATGAAGAAAGCCTATTATCTGACCCAGAAAATTCCTGATCAGGACT
TCTATGCATCTGTGCTGGATGTCCTGACACCAGATGATGTTCGGATTCTGGTTGAGATGGAAGATGAGTT
TTCTCGCCGTGGTCAGTTTGAACGAATTTTTCCTTCTCATATCTCCTCTCGCTATCTCCGCTTTTTTGAG
CAGCCACGATATTTCAACATTCTCACCACCCAATGGGAACAGAAATACCATGGCAACAAGCTTAAAGGAG
TAGATCTGCTCCGGAGTTGGTGCTACAAAGGGTTCCACATGGGAGTTGTCTCTGATTCTGCTCCAGTGTG
GTCTCTCCCGACATCACTTCTGACTATCTCAAAGGATGACGTGATACTCAATGCCTTCAGCAAATCAGAG
ACTAGCAAGCTGGGAAAACAAAGCTCCTGTGAGGTTAGCCTACTACTCTCTGAAGACGGGACCACGCCCA
AATCCAAGAAGACTCAAGCTGGCCTTTCCCCTTATCCCCAGAAACCCAGTTCCTCAAAGGACAGTGAGGA
CACCAGCAAAGAGCCCAGCCTTTCTACCCAGACGTTACCTGTGATCAAGTGCTCTGGGCAGACTTCAAGA
CTTTCTGCTTCCTCCACTTTCCAGTCAATCAGTGACTCCCTCCTGGCTGTGAGCCCATAACTGGCCTCTC
TCCAAAAGCCTCTGCCCAGGAGCATGGGCATCAGCTACCTCACGGGAACCAGCCTGCTGTTCAGACCAGT
CTGACCCCCTACCCCTTTCACCCTGTCCCTCCTCAGAGTATTTTTTGAAGTGGTTGCATTATAGAGATGG
GTATTTGTAGGGCCGGAGGGATGGTAGTGATGGGGAGAAGGTGAGGAAGGGTCACCCTCTGTCACCTGTC
TGCCTGGCTGGCACCTCATATCTCAGCAGAGAAGCCAGTGGTGGCCACGCAGCCTTATAAAGCAGGTTTT
GGTTTCTACCTTAAGTGAGCCATGTGTGGTTTGTCTGGGGGCCCTGGTGTGGTTGCTGAGTTGTAGCTCA
AGAGGAGAAAACATACAGAACATATTTGGACCGGAAATCCTTTGTTCTGAATTTGAGGGGGTCTTCTGAG
GTCCTTACTTCCTTAGGTCTTTCCTCACCCCTCTCCCACCGCTGTCCTGAGGAGAAACCCTTGAACTTCC
TCAGTAGACAGGCGGAGAGGCCACAACATGCCGAACCCATTTCCTGTCATCCTAGTCTTGGGTCTTCACC
GCCTCCTTCCAAATACCCACCCTGCCAGCAGCCCTAGGTCTTCCTGTTCTGACCCCCCATCACTGCTCGT
TCAGCCTTCTAGATGTCTCTCTCGTGGACATCTGTTCTTTAGCTGTTGGCTTTCTCTGAGGTGTGAGAGG
GTCTATGAACTTTGTGAATTTCCCATGGCCCCAGTGAAGGAGCCCAGATAATCCCAGTAGCTGTTACCTG
TCTCCATGTATCAAAGGACACAGTCCAGGGGGAGGGTGGAAGGAGATGTGGTTTCTCTATAGTGCAACAA
ACATGGTTTCTCAATGTTCTGCTGTGCAGCAAGCAGGGTCTGGCGGCTTGGTAGGTGGGTTTCAGGAGCA
GTCACTATTGTAGGATGGGCTTCCAATCAAACCTCAGACTAAACTCTTGTACTGAACTGATTCTACCTCC
CTCCTCTAGACTCAGTAAACAGTGACTATTCAATAAA

The term “TTLL4” is intended to include fragments, variants (e.g., allelic variants), and derivatives thereof. Representative human TTLL4 cDNA and human TTLL4 protein sequences are well-known in the art and are publicly available from the National Center for Biotechnology Information (NCBI). For example, at least five different human TTLL4 isoforms are known. Human TTLL4 isoform X1 (XP_016860876.1) is encodable by the transcript variant X1 (XM_017005387.2). Human TTLL4 isoform X2 (XP_047302407.1) is encodable by the transcript variant X2 (XM_047446451.1). The amino acid sequences provided above are also for the X2 variant. Human TTLL4 isoform X3 (XP_047302410.1) is encodable by the transcript variant X3 (XM_047446454.1). Human TTLL4 isoform X4 (XP_047302411.1) is encodable by the transcript variant X4 (XM_047446455.1). TTLL4 isoform X5 (XP_047302415.1) is encodable by the transcript variant 5 (XM_047446459.1). Nucleic acid and polypeptide sequences of TTLL4 orthologs in organisms other than humans are well known and include, for example, chimpanzee TTLL4 (XM_009444325.2-XP_009442600.1, XM_009444326.2-XP_009442601.1, XM_016950529.1-XP_016806018.1, XM_009444320.2-XP_009442595.1, XM_001159133.4-XP_001159133.1, XM_024355083.1-XP_024210851.1, XM_016950528.1-XP_016806017.1, XM_009444327.2-XP 009442602.1, XM 009444328.3-XP_009442603.1, XM_016950525.1-XP_016806014.1, XM 016950526.1-XP_016806015.1, XM_016950527.1-XP_016806016.1, XM_009444321.2-XP_009442596.1, and XM_009444322.2-XP_009442597.1), monkey TTLL4 (XM 015111085.2-XP 014966571.2, XM 001094864.4-XP 001094864.2, XM 015111089.2-XP_014966575.2, XM_015111086.2-XP_014966572.2, XM 028831122.1-XP_028686955.1, XM_028831123.1-XP_028686956.1, XM 015111088.2-XP_014966574.2), dog TTLL4 (XM_038447958.1-XP_038303886.1, XM 038447959.1-XP 038303887.1, XM_038447960.1-XP_038303888.1), cattle TTLL4 (NM_001205708.2-NP_001192637.2), mouse TTLL4 (XM_006496224.3-XP 006496287.1, XM_006496225.2-XP_006496288.1), rat TTLL4 (XM_039084643.1-XP_038940571.1, XM 001074653.6-XP_001074653.1, XM_039084644.1-XP 038940572.1, XM_017596826.2-XP_017452315.1), chicken TTLL4 (XM_040703867.2 XP 040559801.1, XM_040703868.2-XP_040559802.1, XM_040676618.2-XP 040532552.1, XM_040676619.2-XP_040532553.1), tropical clawed frog TTLL4 (XM_002933944.5→XP_002933990.3), and C. elegans TTLL4 (NM_001027814.4-NP_001022985.1, NM_001027815.6-NP_001022986.1).

Anti-TTLL4 antibodies suitable for detecting TTLL4 protein are well-known in the art and include, for example, antibodies AP54410PU-N and TA321025 (Origene), antibodies NBP1-81535, NBP1-47243, and NBP2-20756, (Novus Biologicals, Littleton, CO). In addition, reagents are well-known for detecting TTLL4. Multiple clinical tests of TTLL4 are available in NIH Genetic Testing Registry (GTR®)(e.g., GTR Test ID: GTR000544836.3, offered by Fulgent Clinical Diagnostics Lab (Temple City, CA)). Moreover, multiple siRNA, shRNA, CRISPR constructs for reducing TTLL4 expression can be found in the commercial product lists of the above-referenced companies, such as siRNA products #sc-94714 and sc-154792 and CRISPR products #sc-408173, sc-408173-NIC, sc-408173-NIC-2, sc-408173-HDR, sc-408173-ACT, sc-408173-ACT-2, sc-408173-LAC, and sc-408173-LAC2 (Santa Cruz, Dallas, TX), CRISPR products #487291110191, 487291110195 and #487291110101 from ABM (Richmond, BC Canada), and CRISPR products #GA106471, GA207385, KN405206, and KN518445 (Origene), CRISPR products #Gene Knockout Kit v2-human-TTLL4 (Synthego, Menlo Park, CA), and CRISPR products #sc-408173, sc-408173-NIC, sc-408173-NIC-2, sc-408173-HDR, sc-408173-ACT, sc-408173-ACT-2, sc-408173-LAC, and sc-408173-LAC2 (Santa Cruz). It is to be noted that the term can further be used to refer to any combination of features described herein regarding TTLL4 molecules. For example, any combination of sequence composition, percentage identify, sequence length, domain structure, functional activity, etc. can be used to describe a TTLL4 molecule encompassed by the present invention.

Numerous methods for detecting a nucleic acid or a protein are well known in the art. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled antibody against the desired protein to be detected. For example, the antibody can be labeled with a radioactive marker whose presence and location in a subject can be detected by standard imaging techniques.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

### Compositions

The present invention involves inhibiting the expression or activity of TTLL4, especially its ability to catalyze glutamate-glutamylation of NPM1c. Accordingly, compositions are provided comprising an inhibitor of TTLL4 or a gene editing system for modifying the TTLL4 gene such that its expression and/or activity is reduced or eliminated.

In some embodiments, the inhibitor of TTLL4 is a small molecule, an inhibitory nucleic acid (e.g., siRNA, miRNA, shRNA, antisense oligonucleotide (ASO), aptamer, and the like), or an antibody, or a fragment thereof, that specifically binds TTLL4. In some embodiments, the inhibitor is a time-dependent inhibitor (see, e.g., Mahalingan et al., Nat. Structural & Mol. Bio., 27:802-813 (2020).

TTLL4 expression and/or activity can be modulated (i.e., reduced or eliminated) by a small molecule inhibitor. The ability of a small molecule inhibitor of TTLL4 can be tested in vitro using any method known in the art or described herein. Methods for making small molecules are known in the art (see, e.g., sulfur (VI) fluoride exchange-enabled high-throughput medicinal chemistry (Kitamura et al., J. Am. Chem. Soc., 142:10899-10904 (2020)).

In some embodiments, a small molecule inhibitor useful in the present methods has the structure of Formula (I):

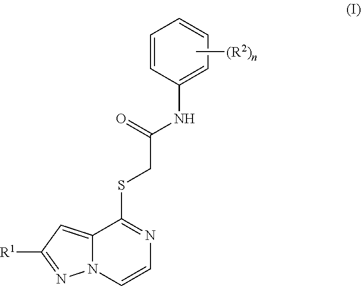

or a pharmaceutically acceptable salt thereof, wherein:
R1 is (C₁-C₁₀) aryl, which is optionally substituted with one to three substitutents selected from (C₁-C₆)alkyl, (C₁-C₆) fluoroalkyl, (C₁-C₆)alkoxy and chloro;
R² is independently for each occurrence (C₁-C₆)alkyl, (C₁-C₆)alkoxy, (C₁-C₆)thioalkoxy, acetyl, cyano, fluoro, and chloro; and
n is 0, 1, 2, or 3.

In certain embodiments, R¹ is phenyl optionally substituted with one to three substitutents selected from (C₁-C₆)alkyl, (C₁-C₆)alkoxy and chloro. In more particular embodiments, R¹ is phenyl, 4-methylphenyl, 4-chlorophenyl, 4-ehtylphenyl, 4-methoxyphenyl, 2-methoxyphenyl, 4-butoxyphenyl, or napthalen-2-yl.

In some embodiments, R² is independently for each occurrence selected from methyl, ethyl, isopropyl, chloro, fluoro, cyano, methoxy, acetyl, thiomethoxy, and trifluoromethyl.

In some more specific embodiments, n is 0, and in further embodiments, n is 1, while in other embodiments, n is 2, and in still other embodiments, n is 3.

In some embodiments, of a compound of Formula (I),

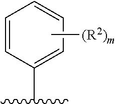

is:
phenyl, 4-methylphenyl, 2,4,6-trifluoromethylphenyl, 4-ethylphenyl, 2-methylphenyl, 2,6-dimethylphenyl, 2,5-dimethylphenyl, 2,4-dimethylphenyl, 4-isopropyl, 3,4-dimethylphenyl, 3-chlorophenyl, 2-chlorophenyl, 4-chlorophenyl, 2-chloro-4-methylphenyl, 2-methyl-4-chlorophenyl, 2-methyl-3-chlorophenyl, 3-fluorophenyl, 4-fluorophenyl, 2,4-difluorophenyl, 2-methyl-5-fluorophenyl, 4-methyl-5-fluorophenyl, 2,5-difluorophenyl, 3-methoxyphenyl, 2-methoxyphenyl, 4-methoxyphenyl, 2,4-dimethoxyphenyl, 2,5-dimethoxyphenyl, 3,4-dimethoxyphenyl, 3-thiomethoxyphenyl, 2-trifluormethylphenyl, 4-acetylphenyl, 2-trifluoromethyl-4-chlorophenyl, and 3-cyanophenyl.

An exemplary synthesis of a compound of formula (1) is shown below. Those skilled in the art may readily adapt this scheme to prepare additional compounds of formula (I).

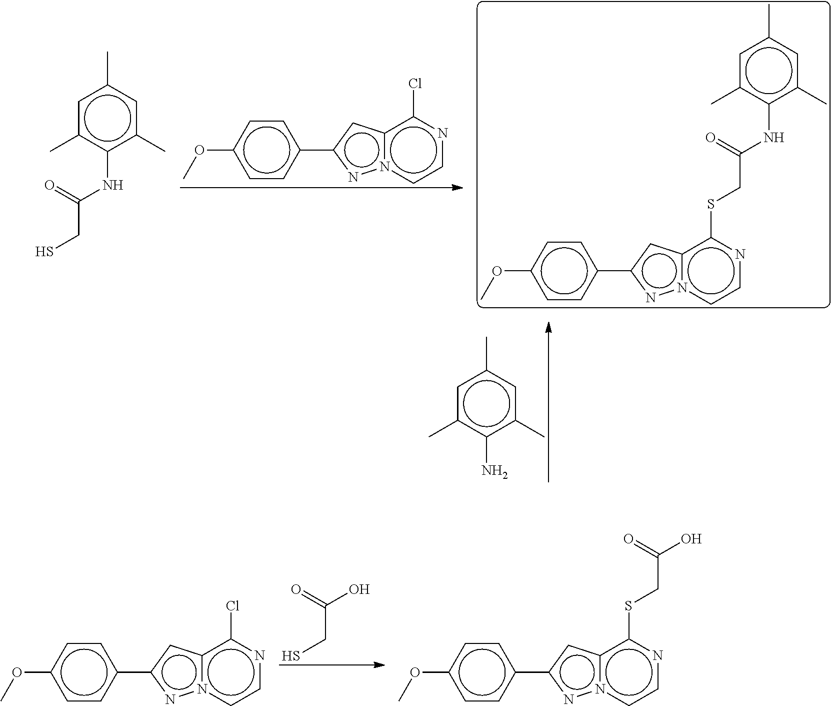

### Scheme 2

In other embodiments, the small molecule inhibitor has the structure of Formula (II):

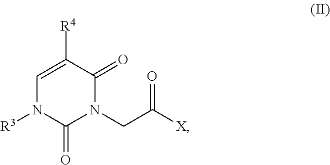

or a pharmaceutically acceptable salt thereof, wherein:
   R³ is (C₃-C₅) cycloalkyl, 4- to 7-membered hetercyloalkyl, and 5- to 6-membered heteraryl;
   R⁴ is hydrogen, fluoro, (C₁-C₆)alkyl, or (C₁-C₆) haloalkyl;
X is

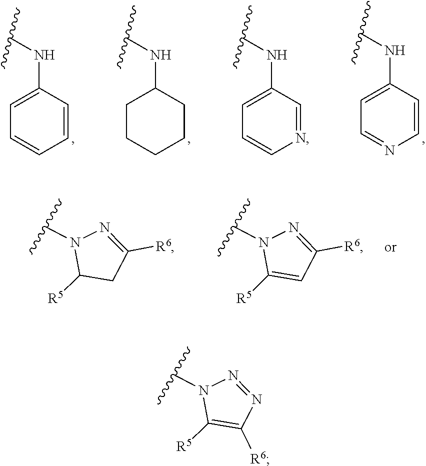

and
   R⁵ and R⁶ are each independently hydrogen, furanyl, thiophenyl, or phenyl, wherein phenyl is optionally substituted with one, two or three substituents selected independently from fluoro, chloro, bromo, hydroxy, (C₁-C₆)alkoxy, and NH₂.

In some embodiments, R³ is cyclopentyl, tertrahydrofuran-3-yl, tetrahydrothiophen-3-yl, tetrahydrothiophen-2-yl, furan-2-yl, pyrrolidine-2-yl, or pyrrol-2-yl.

In some more particular embodiments, R⁴ is fluoro.

In certain embodiments, R⁵ is thiophen-2-yl, furan-2-yl, phenyl, 4-chlorophenyl, or hydrogen.

In some embodiments, R⁶ is 4-chlorphenyl, 4-fluorophenyl, 4-bromophenyl, 4-hydroxyphenyl, 4-aminophenyl, phenyl, 4-methoxyphenyl, or hydrogen.

In more particular embodiments, X is:

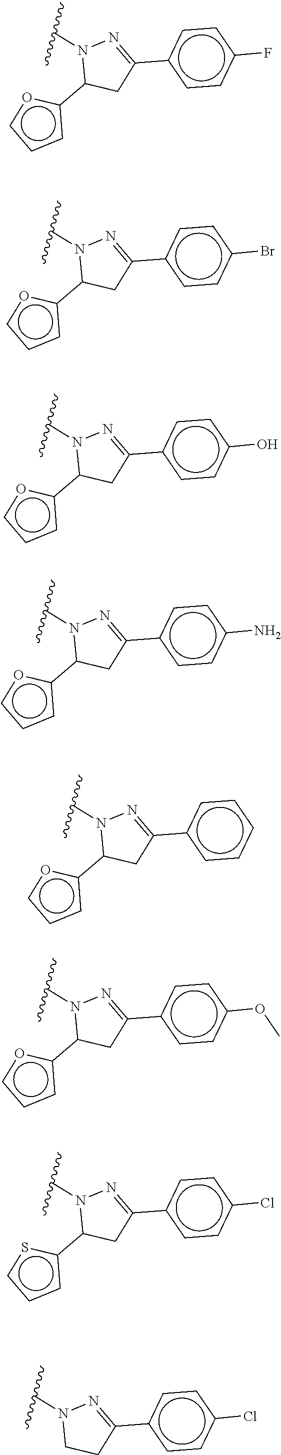

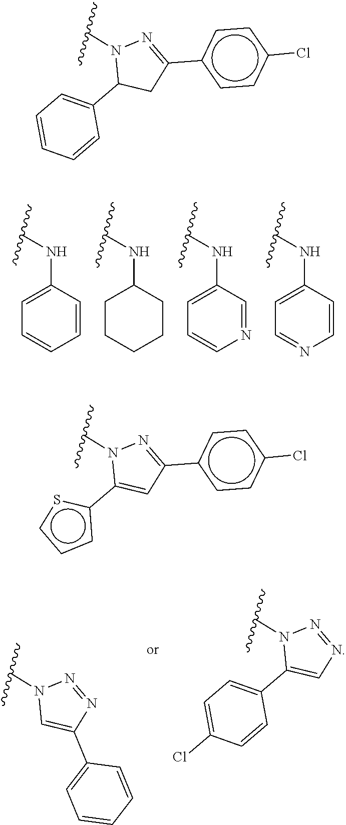

An exemplary synthesis of a compound of formula (II) is shown below. Those skilled in the art may readily adapt this scheme to prepare additional compounds of formula (II).

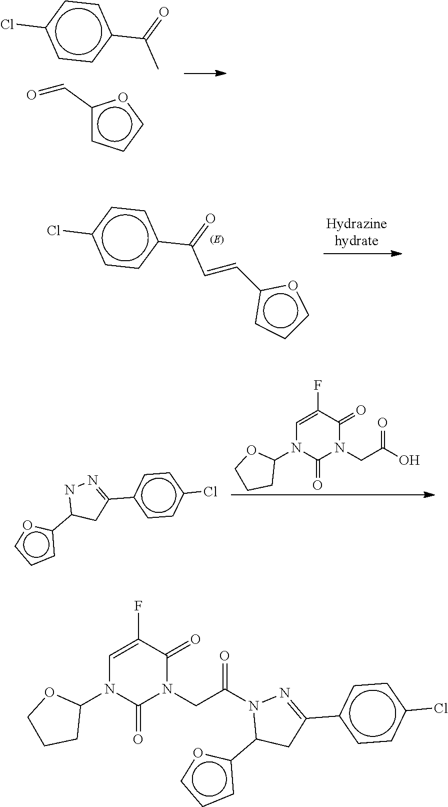

In certain embodiments, the small molecule inhibitor is:

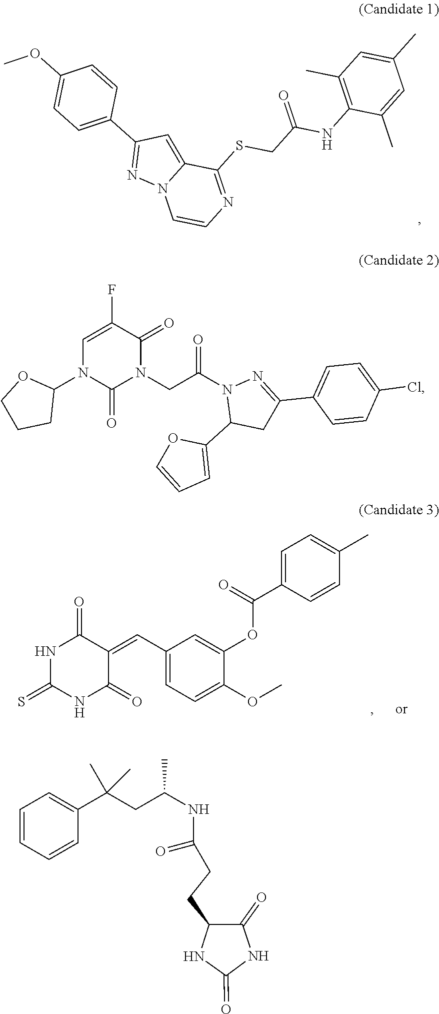

or a pharmaceutically acceptable salt thereof.

TTLL4 expression and/or activity can be modulated (i.e., reduced or eliminated) by an inhibitory nucleic acid such as an siRNA, miRNA, shRNA, ASO, or an aptamer. For example, siRNAs are short twenty-one to twenty-five nucleotide double-stranded RNAs effective at down-regulating gene expression (Zamore et al., Cell 101:25-33; Elbashir et al., Nature 411:494-498, 2001, hereby incorporated by reference). The therapeutic effectiveness of an siRNA approach in mammals was demonstrated in vivo by McCaffrey et al. (Nature 418:38-39.2002).

The inhibitory nucleic acid molecules of the present invention may be employed as double-stranded RNAs for RNA interference (RNAi)-mediated knock-down of expression. In one embodiment, RNAi is used to reduce or eliminate expression of TTLL4 in a cell. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). Given the sequence of a target gene, siRNAs may be designed to inactivate that gene. Such siRNAs, for example, could be administered directly to an affected tissue, or administered systemically. The nucleic acid sequence of a gene can be used to design small interfering RNAs (siRNAs). The 21 to 25 nucleotide siRNAs may be used, for example, as therapeutics to treat cancer. The introduction of siRNAs into cells either by transfection of dsRNAs or through expression of siRNAs using a plasmid-based expression system is increasingly being used to modulate expression of genes in mammalian cells.

In one embodiment of the invention, a double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh) RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002.

Another inhibitory nucleic acid molecule, small hairpin RNAs (shRNAs), comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. These small regulatory RNAs may repress the translation of target mRNAs or inhibit expression of the protein through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990). The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Catalytic RNA molecules or ribozymes that include an antisense sequence of the present invention can be used to inhibit expression of a nucleic acid molecule in vivo (e.g., a nucleic acid molecule encoding TTLL4). The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591 (1988), and U.S. Patent Application Publication No. 2003/0003469 A1, which is incorporated herein by reference.

Accordingly, the invention also features a catalytic RNA molecule that includes, in the binding arm, an antisense RNA having between eight and nineteen consecutive nucleobases that are at least partially complementary to a nucleic acid sequence in the TTLL4 gene. In some embodiments, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18:299, 1990. These specific motifs are not limiting in the invention and those skilled in the art will recognize that all that is important in an enzymatic nucleic acid molecule of this invention is that it has a specific binding site that is complementary to one or more of the target gene RNA regions, and that it has nucleotide sequences within or surrounding that substrate binding site that impart an RNA cleaving activity to the molecule.

In some embodiments, the composition comprises a site-directed nuclease (e.g., a CRISPR nuclease) and a guide RNA (gRNA), wherein the gRNA hybridizes with a target sequence in the TTLL4 gene in a cell. When the composition comprises a CRISPR nuclease and a gRNA, the composition is said to comprise a CRISPR system. In general, gRNA are between 17 and 24 nucleotides and are at least 80%, 85%, 90%, or 95% identical to a nucleic acid sequence that is complementary to the target sequence. The gRNA can be identical to a nucleic acid sequence that is complementary to the target sequence. In some embodiments, the composition comprises at least one, two, three, four, five, six, seven, eight, nine, or ten gRNA. In some embodiments, the target sequence may be 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% identical to a nucleotide sequence present in SEQ ID NO: 2.

The site-directed nuclease can be a CRISPR system comprising a CRISPR nuclease and a guide RNA (gRNA). The CRISPR nuclease can be a Cas nuclease such as, but not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas9 enzyme is S. pneumoniae, S. pyogenes, or S. thermophilus Cas9, and may include mutated Cas9 derived from these organisms. The Cas9 enzyme may be a nickase or a cleavase. For example, a Cas9 nickase may comprise a D10A mutation. The enzyme may be a Cas9 homolog or ortholog. In some embodiments, the Cas9 protein is codon optimized for expression in the cell.

In some embodiments, the TTLL4 inhibitor is an antibody. Antibodies that specifically bind TTLL4 are useful in the methods of the invention, including therapeutic methods. Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. As used herein, the term “antibody” means not only intact antibody molecules, but also fragments of antibody molecules that retain immunogen-binding ability. Such fragments are also well known in the art and are regularly employed both in vitro and in vivo. Examples of antibody fragments include, but are not limited to, Fab, Fab′, F(ab′)₂, and Fv fragments, linear antibodies, scFv antibodies, single-domain antibodies, such as camelid antibodies (Riechmann, 1999, Journal of Immunological Methods 231:25-38), composed of either a VL or a VH domain which exhibit sufficient affinity for the target, and multispecific antibodies formed from antibody fragments.

The antibodies in the present invention may exist in a variety of forms including, for example, polyclonal antibodies, monoclonal antibodies, Fv, Fab and F(ab′) 2, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). For example, F(ab′) 2, and Fab fragments that lack the Fc fragment of an intact antibody, clear more rapidly from the circulation, and may have less nonspecific tissue binding than an intact antibody (Wahl et al., J. Nucl. Med. 24:316 325 (1983). Thus, the antibodies of the invention comprise, without limitation, whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8 (10): 1057-1062,1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake. See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and using unconventional antibodies have been described. Bispecific antibodies produced using leucine zippers are described by Kostelny et al. (J. Immunol. 148 (5): 1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including VH- and VL-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In various embodiments, an antibody is monoclonal. Alternatively, the antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′) 2” fragment, retains both of the antigen binding sites of the intact antibody. Similarly, an antibody from which the Fc region has been enzymatically cleaved, or which has been produced without the Fc region, designated an “Fab”′ fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Methods of preparing antibodies are well known to those of ordinary skill in the science of immunology. Antibodies can be made by any of the methods known in the art utilizing a soluble polypeptide, or immunogenic fragment thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding polypeptides or immunogenic fragments thereof, can be provided to the host in a delivery vehicle that is taken up by immune cells of the host. The cells will in turn express the polypeptide thereby generating an immunogenic response in the host. Alternatively, nucleic acid sequences encoding human polypeptides or immunogenic fragments thereof, can be expressed in cells in vitro, followed by isolation of the polypeptide and administration of the polypeptide to a suitable host in which antibodies are raised.

Alternatively, antibodies may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

In some embodiments, the TTLL4 inhibitor is an aptamer. Aptamers are a class of binding agent or capture reagent that can be used to target the TTLL4 in a cell. Aptamers are nucleic acid-based molecules that bind specific ligands. Aptamers that bind to TTLL4 may reduce or eliminate the biological activity of the protein sufficiently so as to reduce glutamate-glutamylation of NPM1c. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

Nucleic acid molecules encoding inhibitors, such as those described herein, or CRISPR systems can be delivered to cells using a suitable vector. In general, and throughout this specification, the term “vector” refers to a nucleic acid molecule into which another nucleic acid molecule is incorporated. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides that encode proteins necessary for transfection of a host cell.

Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Plasmids can also be expression vectors.

Recombinant expression vectors can comprise a nucleic acid of the presently disclosed subject matter in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed.

### Pharmaceutical Compositions

In another aspect, the present invention provides pharmaceutically acceptable compositions that comprise a therapeutically-effective amount of an agent that modulates (e.g., decreases) biomarker expression and/or activity, formulated together with one or more pharmaceutically acceptable carriers (additives) and/or diluents. As described in detail below, the pharmaceutical compositions encompassed by the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) biomarker expression and/or activity which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods encompassed by the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods encompassed by the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions encompassed by the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents encompassed by the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods encompassed by the present invention so as to obtain an amount of the active ingredient, which is effective to achieve the desired therapeutic response for a particular subject, composition, and mode of administration, without being toxic to the subject.

The nucleic acid molecules encompassed by the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The present invention also encompasses kits for detecting and/or modulating biomarkers described herein. A kit encompassed by the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### Small Molecules

### Methods

As demonstrated herein, TTLL4 catalyzes glutamate-glutamylation of NPM1 (e.g., NPM1c) and is an important factor in cellular differentiation and proliferation. Unregulated TTLL4 activity and/or glutamate-glutamylation of NPM1c can be a characteristic of certain cancers, while inhibiting such activity can improve survival of a subject afflicted with cancer. Accordingly, the present disclosure provides methods for identifying agents (e.g., small molecules, polynucleotides, such as inhibitory nucleic acid molecules, polypeptides, including but not limited to antibodies, including recombinant antibodies,) useful for inhibiting TTLL4 expression and/or activity and/or treating or preventing a disease or disorder characterized by a overexpression of TTLL4 or glutamlyation of NPM1 or NPM1c. For example, models of TTLL4 have been developed as described herein that can be used to identify small molecules, nucleic acid molecules, and peptides that bind to or otherwise interact with a TTLL4 nucleic acid or protein in a manner that reduces the expression and/or activity of TTLL4. Algorithms have been, and continue to be, developed to increase the likelihood that a candidate agent will perform as predicted. For example, as demonstrated herein, there are four potential binding pockets on TTLL4. certain residues on TTLL4 reside in potential binding sites for small molecule inhibitors. The residues include 666, 1719, K721, R727, G728, Q749, R750, Y751, L752, K762, D764, R766, R788, H807, L808, T809, N810, Y811, S812, K815, K833, D893, M895, L905, E906, 1909, S912, H914, D920, and K924 of TTLL4. Without being bound by theory, it is believed that the F666 and 1909 residues are important for the specificity of any candidate molecule that targets TTLL4. In some embodiments, once a candidate agent is found, the method further comprises synthesizing the agent.

Once a candidate agent is identified and synthesized, it can be screened in vitro to characterize its activities and to assess its potential for laboratory or therapeutic uses. Screens candidate agents described herein can include, but are not limited to, TTLL4 enzyme assays that characterize TTLL4 activity, for example, an assay comprising a cell sample in which the cells comprise a wild type TTLL4 protein or a nucleic acid molecule encoding the wild type TTLL4 protein. The use of such cells, which express TTLL4 in normal conditions (i.e., in the absence of inhibitor), is particularly advantageous for the identification of agents that decrease TTLL4 expression or biological activity. Methods of observing changes in TTLL4 biological activity (e.g., glutamate-glutamylation of NPM1 or NPM1c) can be exploited in high throughput assays for the purpose of identifying compounds that modulate TTLL4 biological activity, e.g., transcriptional regulation or protein-nucleic acid interactions.

Any number of methods are available for screening new candidate compounds that reduce or eliminate the expression or activity of TTLL4. In one example, candidate compounds (e.g., small molecules) are added at varying concentrations to the culture medium (e.g., medium comprising methylcellulose when the cells comprise a gene encoding NPM1c) of cultured cells expressing TTLL4. In various embodiments, the cell is an hematopoietic cell. In some embodiments, the cell is an AML cell characterized by NPM1c expression. Gene expression is then measured, for example, by microarray analysis, Northern blot analysis (Ausubel et al., supra), or RT-PCR, using an appropriate hybridization probe. The level of gene expression in the presence of the candidate compound is compared to the level measured in a control culture medium lacking the candidate molecule. A compound which reduces or eliminates the expression of a TTLL4 gene, or a functional equivalent thereof, is considered useful in the invention; such a molecule may be used, for example, as a therapeutic to treat a human subject having or suspected of having a cancer characterized by overexpression of TTLL4 (e.g., renal cell kidney cancer and melanoma) or AML characterized by NPM1c expression.

In another example, the effect of candidate compounds may be measured at the level of polypeptide production using the same general approach and standard immunological techniques, such as Western blotting or immunoprecipitation with an antibody specific for a polypeptide encoded by a TTLL4 gene. For example, immunoassays may be used to detect or monitor the expression of at least one of the polypeptides of the invention in an organism. Polyclonal or monoclonal antibodies that are capable of binding to such a polypeptide may be used in any standard immunoassay format (e.g., ELISA, Western blot, or RIA assay) to measure the level of the polypeptide. In some embodiments, a compound that promotes a reduction or elimination of the expression or biological activity of TTLL4 is considered particularly useful. Again, such a molecule may be used, for example, as a therapeutic to delay, ameliorate, or treat a cancer in a human subject.

In yet another working example, candidate compounds may be screened for those that specifically bind to a polypeptide encoded by a TTLL4 gene. The efficacy of such a candidate compound is dependent upon its ability to interact with such a polypeptide or a functional equivalent thereof. Such an interaction can be readily assayed using any number of standard binding techniques and functional assays. In some embodiments, a candidate compound is tested for its ability to reduce or eliminate the biological activity of TTLL4 (i.e., glutamate-glutamylation of NPM1c) or NPM1c. The biological activity of a TTLL4 polypeptide may be assayed using any standard method.

In another example, a candidate compound that binds to a polypeptide encoded by a TTLL4 gene may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the TTLL4 polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to reduce or eliminate the activity of a TTLLA polypeptide (e.g., as described herein). Compounds isolated by this approach may also be used, for example, as therapeutics to treat cancer in a human patient.

Animal models may also be used to screen candidate compounds. For example, methods of generating genetically modified animals having mutations (e.g., in TTLL4) in organisms are known in the art and available to the ordinarily skilled person. In various embodiments, a CRISPR-Cas9 system is used to create a genetically modified organism (see e.g., U.S. Pat. Nos. 8,771,945 and 8,945,839, and US Patent Publication Nos. 20140170753, 20140227787, 20150184139, 20150203872, which are herein incorporated by reference in their entirety). Such organisms may include any eukaryotic organism, including, without limitation, zebrafish and mice. Candidate compounds may be tested for their ability to reduce or eliminate TTLL4 activity and/or NPM1c glutamate-glutamylation. Tissues of test organisms can be assayed in a number of ways that are routine and well known, including, without limitation, immunohistochemical staining, in situ hybridization, and electron microscopy.

Potential antagonists include organic molecules, peptides, peptide mimetics, polypeptides, nucleic acids, and antibodies that bind to a nucleic acid sequence or polypeptide of the invention (e.g., a TTLL4 polypeptide or nucleic acid molecule). Candidate compounds can be assayed to determine if they reduce the turnover rate for TTLL4 (e.g., estimated at ˜12 turnovers/minute or that have binding affinities for glutamate and ATP less than 257 μM or 41 μM, respectively.

Also provided herein are methods for modulating TTLL4 expression and/or activity, therapeutic methods, as well as methods of identifying and screening inhibitors of TTLL4 activity.

One aspect of the present invention provides a method for reducing or eliminating the expression and/or activity of TTLL4 in a cell by contacting the cell with an inhibitor of TTLL4. TTLL4 is an enzyme that catalyzes the glutamate-glutamylation of NPM1c, which is associated with cellular proliferation. Accordingly, another aspect of the present invention provides a method of reducing or eliminating glutamate-glutamylation of NPM1c by contacting the cell with an inhibitor of TTLL4, such as an inhibitor described herein or a CRISPR system comprising a gRNA and a site-directed nuclease (e.g., a Cas9 protein). These methods can lead to apoptosis, differentiation, and reduced or eliminated proliferation of a target cell.

Essentially any method for introducing a nucleic acid construct (i.e., a vector) into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. For example, an shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors, comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39:914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11:975-982, for a description of inducible shRNA.

### Therapeutic Methods

In another aspect of the present invention, methods are provided for the treatment and/or prevention of cancer. In these methods, an inhibitor or a CRISPR system comprising a gRNA and a site-directed nuclease are administered to a subject to treat the cancer.

The present invention provides methods of treating disease and/or disorders or symptoms thereof that comprise administering a therapeutically effective amount of a pharmaceutical composition comprising a TTLL4 inhibitor or a CRISPR system targeting TTLL4 described herein to a subject (e.g., a mammal such as a human). Thus, one embodiment is a method of treating a subject suffering from or susceptible to a disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The decreased expression or activity of TTLL4 and/or decreased glutamate-glutamylation of NPM1 (e.g., NPM1c), which is glutamylated by TTLL4, in a cell, promotes cellular differentiation and/or inhibits cellular proliferation. These methods prevent or treat a cancer characterized by increased TTLL4 expression or activity and/or glutamylated NPM1 (e.g., NPM1c). Accordingly, the invention provides for the treatment of a variety of cancer associated with increased TTLL4 expression or activity and/or decreased glutamate-glutamylation of NPM1. Examples of cancers include, without limitation, hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, rhabdosarcoma, serocystic sarcoma, synovial sarcoma, telangiectaltic sarcoma, Hodgkin's Disease, Non-Hodgkin's Lymphoma, multiple myeloma, neuroblastoma, bladder cancer, breast cancer, ovarian cancer, lung cancer, colorectal cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

In some embodiments, the methods and compositions provided herein relate to the treatment of a leukemia. The term “leukemia” is meant broadly progressive, malignant diseases of the hematopoietic organs/systems and is generally characterized by a distorted proliferation and development of leukocytes and their precursors in the blood and bone marrow. Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

In some embodiments, the methods and compositions provided herein relate to the treatment of a carcinoma. The term “carcinoma” refers to a malignant growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

In some embodiments, the methods and compositions provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and compositions described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

Particular categories of tumors that can be treated using methods and compositions described herein include lymphoproliferative disorders, breast cancer, ovarian cancer, prostate cancer, cervical cancer, endometrial cancer, bone cancer, liver cancer, stomach cancer, colon cancer, colorectal cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

Cancers treated in certain embodiments also include precancerous lesions, e.g., actinic keratosis (solar keratosis), moles (dysplastic nevi), acitinic chelitis (farmer's lip), cutaneous horns, Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

The compositions described herein may be delivered by any suitable route of administration, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

The terms “systemic administration,” “administered systemically,” “peripheral administration,” and “administered peripherally” as used herein mean the administration of the composition comprising the dual virus packaging system (i.e., rAAV (e.g., rAAV-Onco-CRISPR or rAAV-TSG) and Ad-rAAVpack)) or the rAAV-Onco-CRISPR or rAAV-TSG, alone, such that it enters the patient's system and, thus, is subject to metabolism and other like processes.

The terms “parenteral administration” and “administered parenterally” as used herein mean modes of administration other than enteral and topical administration, usually by injection, and includes, without limitation, intravenous, intramuscular, intarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection, intratumoral injection, and infusion.

In certain embodiments the pharmaceutical compositions are delivered generally (e.g., via oral or parenteral administration). In certain other embodiments the pharmaceutical compositions are delivered locally through direct injection into a tumor or direct injection into the tumor's blood supply (e.g., arterial or venous blood supply). In some embodiments, the pharmaceutical compositions are delivered by both a general and a local administration. For example, a subject with a tumor may be treated through direct injection of a composition containing a composition described herein into the tumor or the tumor's blood supply in combination with oral administration of a pharmaceutical composition of the present invention. If both local and general administration is used, local administration can occur before, concurrently with and/or after general administration.

Other embodiments encompassed by the present invention are described in the following Examples. The present invention is further illustrated by the following examples which should not be construed as further limiting.

### Examples

### Example 1: Elucidating TTLL4's Role in NPM1 Glutamate-Glutamylation

Post-translational glutamate-glutamylation is found on histone chaperones, including NPM1 (FIG. 2A)(3, 25-30). NPM1 is glutamylated by TTLL4 and glutamate-glutamylation is removed by CCP5 (cytosolic carboxypeptidase-like 5)(FIGS. 2B-2D). TTLL4 and CCP5 both have oncogenic roles (27, 29, 31-34). Consistent with a chromatin regulatory mechanism, NPM2 glutamate-glutamylation enhances its affinity for histones (2).

### Example 2: Target Validation

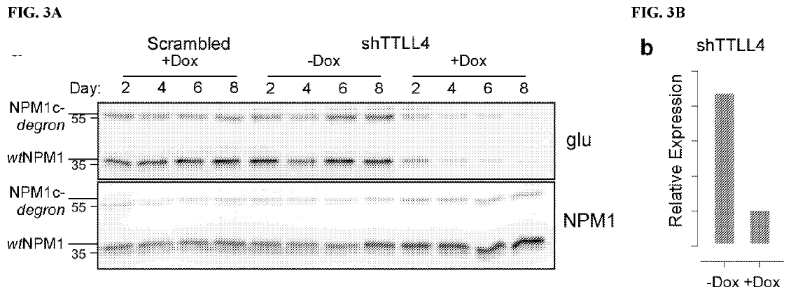

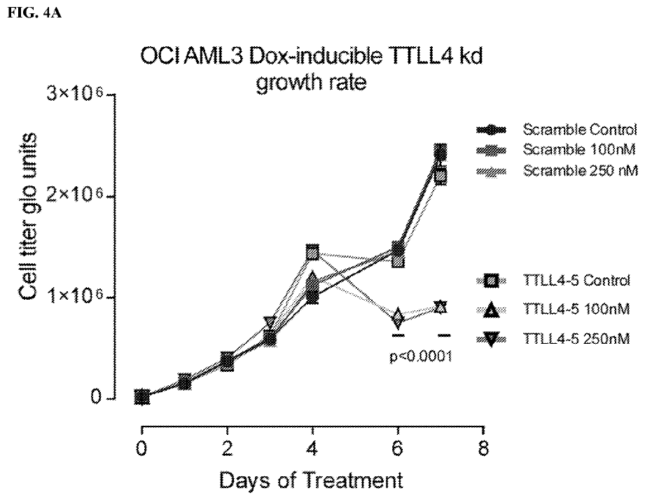

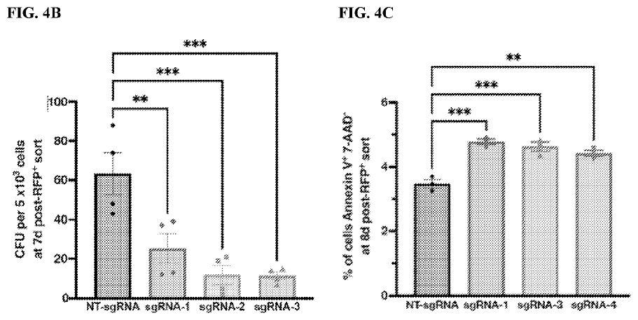

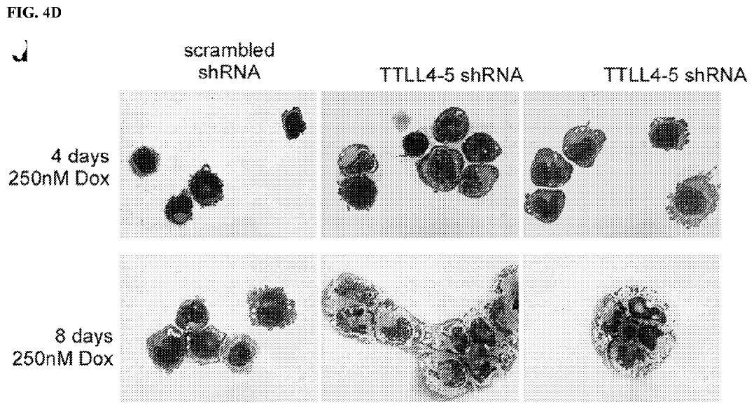

The OCI-AML3 human cell line, which comprises a degron-inducible NPM1c (gift of the Goodell lab (35); FIG. 3A), degradation of the heterozygous NPM1c with dTAG-13 leads to loss of glutamate-glutamylation of the NPM1 wt protein (NPM1/NPM1c forms a heterologous pentamer, FIG. 3A). A doxycycline-inducible TTLL4 shRNA was introduced to this cell line. TTLL4kd eliminated NPM1c glutamate-glutamylation and reduced cellular proliferation (FIGS. 3A, 3B, and 4A). In addition, CRISPR-Cas9 mediated knockout of TTLL4 in OCI-AML3 cells reduced colony formation in methylcellulose (FIG. 4B, methylcellulose colony forming assays are used to detect cell proliferation and hematopoietic/progenitor activity) and increased apoptosis (FIG. 4C). Furthermore, analysis of cellular morphology revealed that TTLL4 knockdown promotes myeloid differentiation (FIG. 4D).

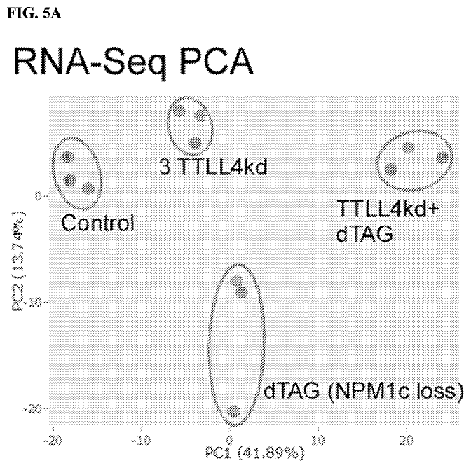

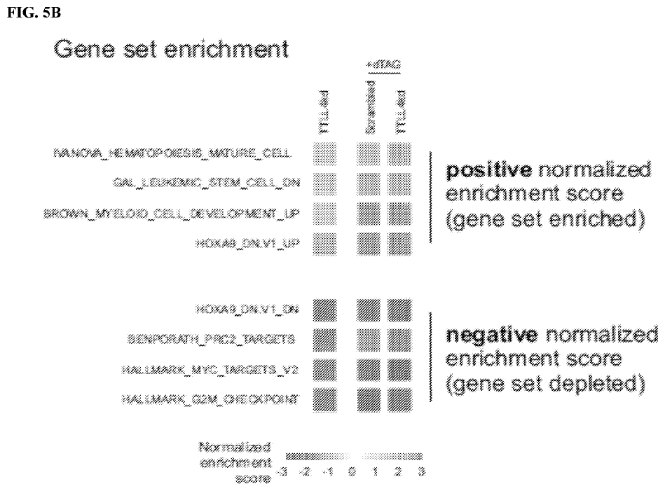

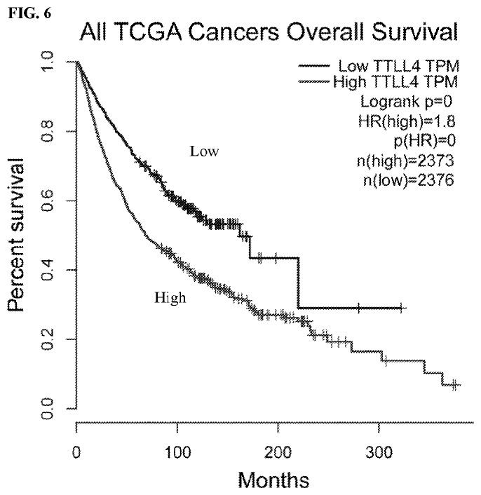

RNA-seq of OCI-AML3 degron cells +/−TTLL4 KD demonstrated that TTLL4 KD results in gene expression signatures similar to those that are caused by NPM1c-degron mediated depletion, which are consistent with hematopoietic differentiation and loss of self-renewal (FIGS. 5A, 5B). Upon NPM1c degradation, decreased expression was also observed of several genes associated with stem cell self-renewal and leukemogenesis, including HOXA/B cluster genes and MEIS1.

Synergistically with NPM1c degradation, TTLL4 knockdown resulted in reduced leukemogenic gene expression, including reduced expression of the HOXA/B cluster genes (FIGS. 5A, 5B). Overall, these results support a role for TTLL4 in the transforming activity of NPM1c and that TTLL4 is a potential therapeutic target for NPM1c AML.

Additionally, as observed in the Cancer Genome Atlas (TCGA), cancers with elevated TTLL4 expression have significantly worse overall survival (FIG. 6); this is specifically true in renal cell kidney cancer and in melanoma. Therefore, there is potential therapeutic targeting of TTLL4 in individual cancers.

### Example 3: TTLL4 Mechanism and Drug Screening Plan

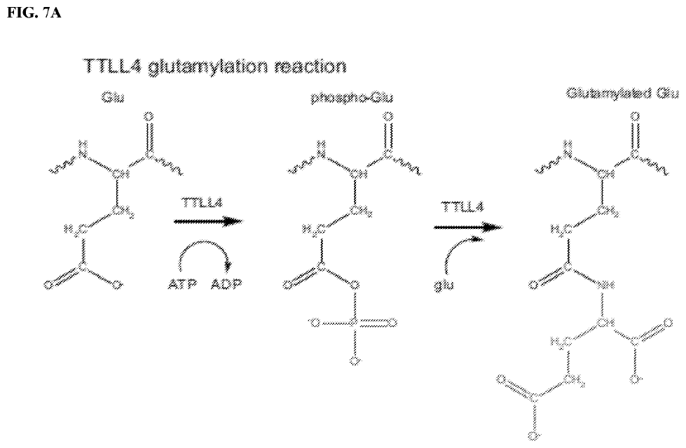

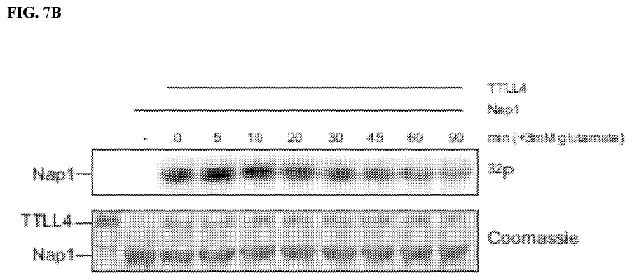

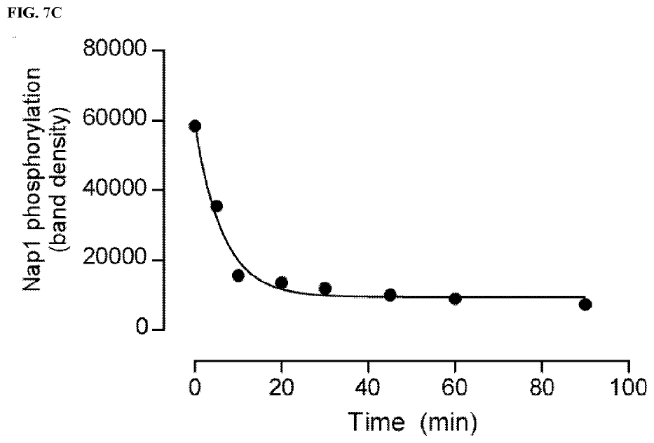

The TTLL family of enzymes has three substrates: ATP, glutamate, and the protein/peptide glutamate acceptor. The enzymatic mechanism of the TTLL family of enzymes is still elusive, so to better understand TTLL4's function, active human TTLL4 enzyme was produced, which was capable of catalyzing in vitro glutamate-glutamylation of Npm1, Npm2, and Nap1 histone chaperone substrates (not shown). This assay confirmed that TTLL4 is a kinase that acts through an acyl-phosphate intermediate (FIG. 7A). Remarkably, the acyl-phosphate is stable, even in acid. TTLL4 then promotes a glutamate reaction with the phospho-glutamate activated carboxylic acid in a nucleophilic acyl substitution reaction (FIGS. 7A-7C). Thus, these three TTLL substrates (ATP, glutamate, and glutamate acceptor) are uniquely targetable ligands for inhibitory drugs.

TTLL4 is a large enzyme with substantial intrinsically disordered regions (IDRs). There is no experimental x-ray or CryoEM structure of TTLL4, but crystal structures have been solved for related enzymes, including TTLL6 (36). TTLL6 was also solved in presence of either ATP (PDB: 6VZT) or of an “initiation intermediate” that mimics the putative transition state of the phosphorylated glutamate (PDB: 6VZW), and these structures are useful modeling resources for TTLL4. The TTLL4 conserved catalytic domain (M551-G1078) is an active enzyme. Google DeepMind Alphafold prediction of this catalytic domain revealed structure similarities to that of TTLL6.

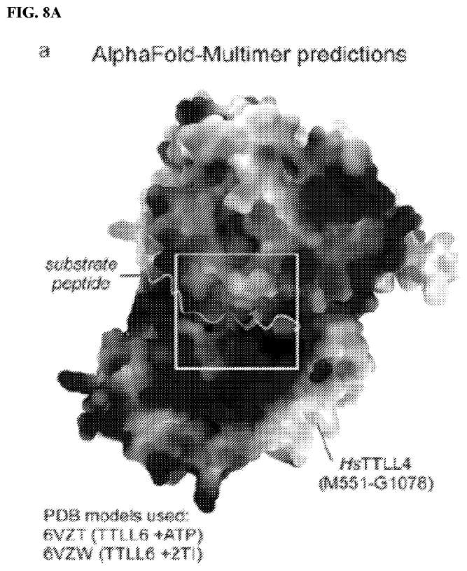

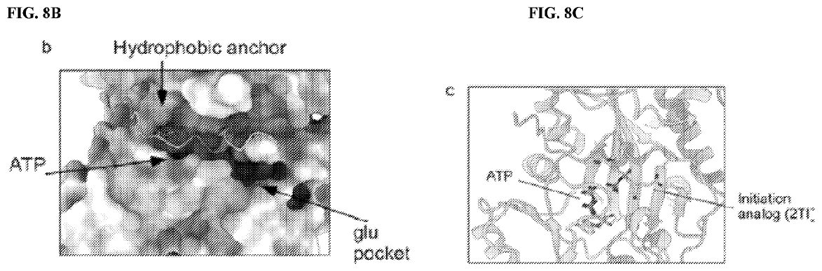

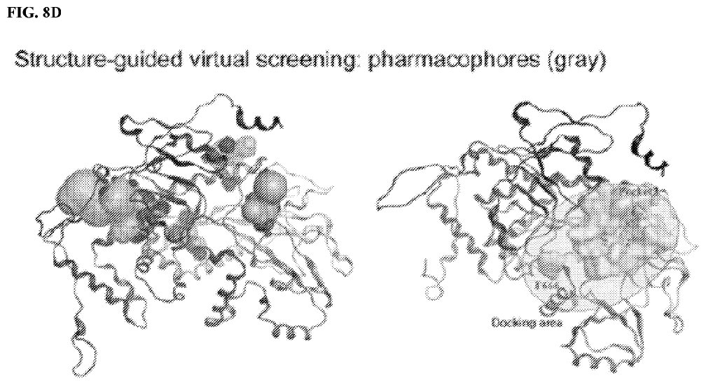

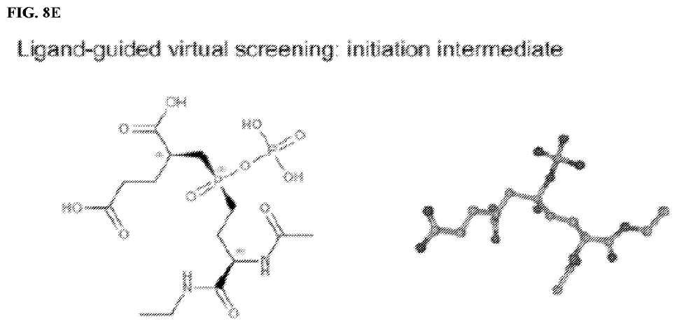

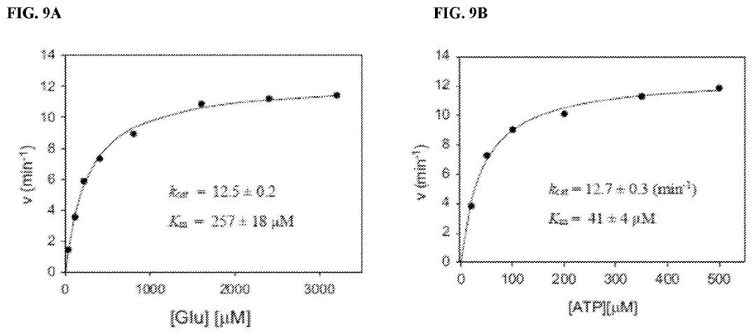

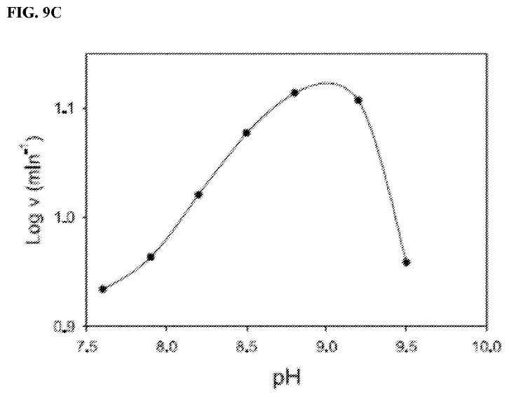

To understand the catalytic mechanism of TTLL4 and to build pharmacophores for drug discovery, Alphafold-multimer (37) was used to build a docked model of TTLL4 catalytic domain with histone chaperone substrates. By using the folded core domain of Nap1 as the alignment and conservation model, Alphafold-multimer produced a compelling TTLL4-substrate bound model (not shown). Similar models were produced for Npm 1 and Npm2; the Nap1 model had the most compelling docking of the acidic IDR substrate, so this model was used for subsequent work. In FIG. 8A, a TTLL4 prediction is shown with just the acidic residue substrate of Nap1. The catalytic pockets, substrates, and ligands are shown in FIGS. 8B and 8C. These pockets were independently found by Schrodinger software, providing support for this approach.

Computational (virtual) screening has been used in 1) Structure-guided screening using the pharmacophore residues identified in FIG. 8D; and 2) ligand-guided screening as shown in FIG. 8E. Hits identified from these screens are assayed in TTLL4 ATPase assays and subjected to additional rounds of virtual screening and SAR to result in lead compounds of at least two chemical classes.

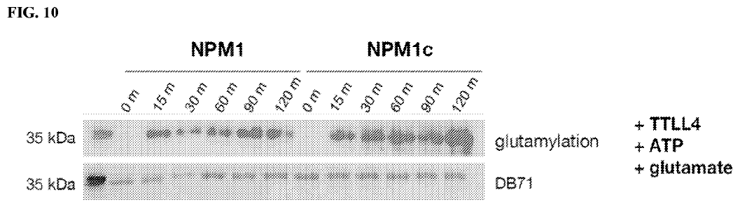

Necessary for characterizing an inhibitor and for determining an effective concentration regime, the enzymatic parameters of TTLL4 were elucidated. As shown in FIG. 10, the enzyme is slow (˜12 turnovers/min) with an ATP and glutamate Km of ˜41 and 257 μM, respectively. Finally, AML-mutant NPM1c is a better in vitro substrate for TTLL4 than the wildtype NPM1 (FIG. 10).

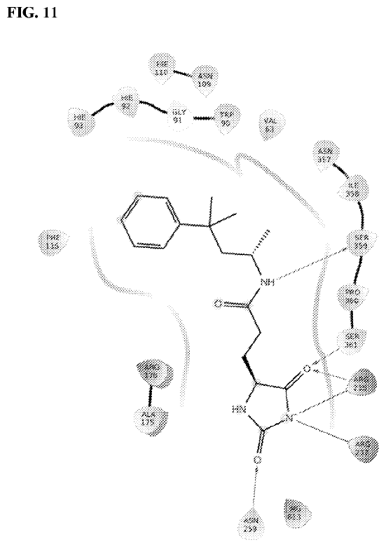

A candidate small molecule inhibitor characterized using the methods described herein is Shown in FIG. 11.

### EXAMPLE 4: Optimization of lead small molecule TTLL4 inhibitors

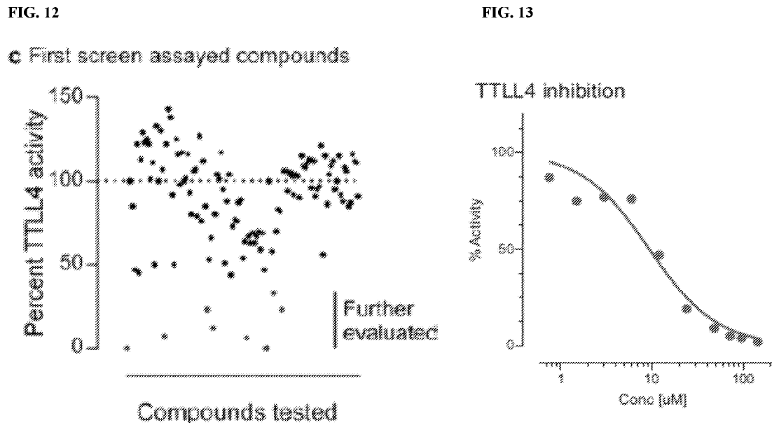

As described above, TTLL4's enzymatic mechanism was characterized, revealing a phospho-glutamate intermediate and a resulting glutamylated-glutamate. We built a TTLL4 enzymatic model and identified pharmacophores to target with small molecules. Using both ligand-based and structure based virtual screening targeting a conserved and highly specific binding pocket on TTLL4, we computationally screened approximately 2 million molecules and identified a diverse library of candidates. Subsequently, structural diversity of the selected molecules was analyzed by fingerprint-based chemical clustering, and the molecules with the highest predicted binding energy in each cluster were selected. Finally, manual inspection of the molecules resulted in over 400 compounds, which we experimentally tested against TTLL4 (FIG. 12) and identified three hit molecules, with low micromolar in vitro IC₅₀ₛ

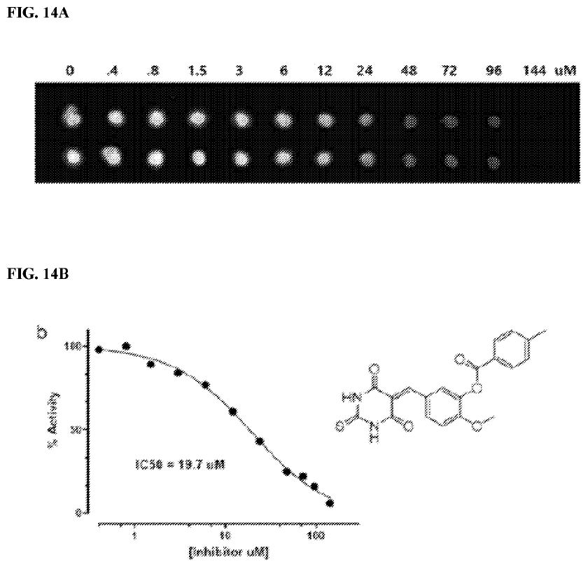

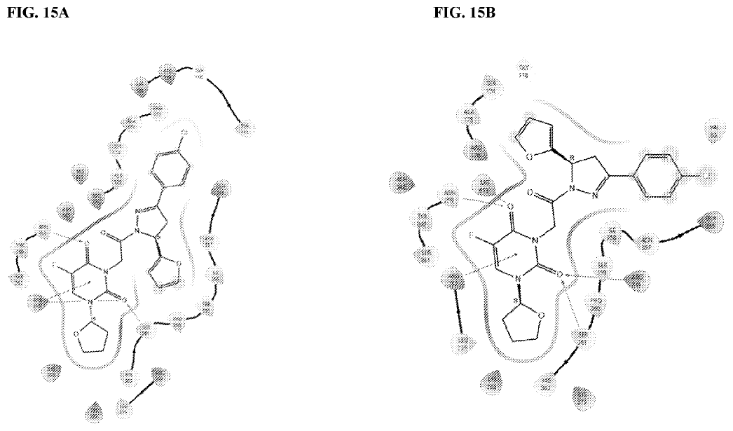

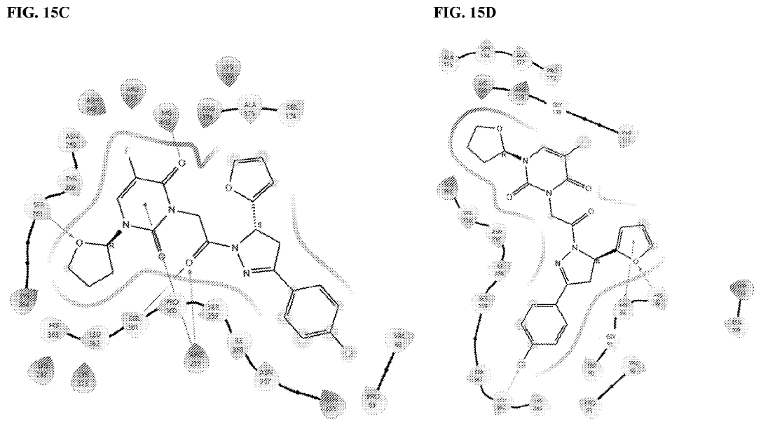

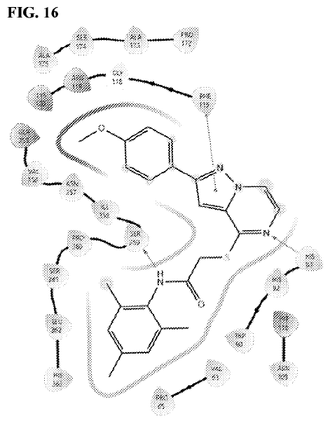

Candidate inhibitors 1 and 2 were evaluated by molecular dynamics docking to the TTLL4 model. Reliable poses and binding energies were found for all the inhibitor enantiomers. Schematics are depicted in FIGS. 15A-d (candidate 2) and FIG. 16 (candidate 1). One docked pose of candidate inhibitor 1 is shown along with the substrates (protein substrate acidic peptide position, ATP, and glutamate), suggesting that the molecule binds between the glutamate and ATP binding sites along the peptide binding ridge. This is consistent with a peptide-competitive inhibitory mechanism, which will be further tested. Crystallography and binding studies are described below. The assay output and an additional candidate inhibitor molecule is shown FIG. 14. Candidates 1, 2, and 3 had an IC50 of 9 μM, 40 μM and 19.7 μM, respectively.

To measure TTLL4 activity and inhibition by lead compounds, we used an immunoassay. The antibody against mono-glutamate-glutamylation was prepared by immunizing white rabbits with the following peptide: MY {Glu (Glu)} DDEEESEAQGPKC (based on studies published here: doi: 10.1016/S0091-679X (10) 95003-6). The serum was purified by cross-adsorption against a non-glutamylated peptide: MYEDDEEESEAQGPKC. The purified antibody was stored in PBS with 0.02% Preclin 300. The final ELISA titer from the purified antibodies against the glutamylated peptide was >1:512,000. These antibodies are noted as “anti-glu”.

To measure activity, recombinant human TTLL4 catalytic domain (composed of residues 561-1199) was produced in E. coli and purified via nickel-affinity and size exclusion column (Superdex 200) chromatography. Similarly, the glutamate-glutamylation substrate Nap2 (gene from Xenopus tropicalis) was produced in E. coli and purified via nickel-affinity size exclusion column (Superdex 200) chromatography. For each assay, 25 micrograms of Nap2 was treated by 1 microgram of TTLL4 catalytic domain in the presence of 0.2 millimolar ATP, 1 millimo-lar glutamic acid, in 50 millimolar Tris-HCl buffer containing 4 millimolar MgCl2, 50 millimolar KCl and 15 milli-molar 2-mercaptoethanol at 30° C. The reaction was stopped at 10 minutes by the addition of 30 millimolar EDTA. Then 1 microliter of the quenched reaction mixture was spotted on 0.45 um nitrocellulose membrane. The membrane was blocked, then incubated for 12 hours in PBS-tween 20 with anti-glu antibody solution. The membrane was washed in PBST, incubated with 1:100,000 anti-rabbit-horseradish peroxidase antibody for 30 minutes. Membrane was then washed, incubated with TMA-6 enhanced chemiluminescence solution (Lumigen ECL Ultra) and imaged on a GE LAS-4000 system. Intensity measurements were quantified with the Microar-ray Profile plugin for FIJI/ImageJ and analyzed with a 4-parameter model in Graphpad Prism v9.

### REFERENCES

1. Papaemmanuil et al. The New England journal of medicine. 2016; 374 (23): 2209-21. Epub 2016 Jun. 9.
2. Warren et al. Nature Communications. 2017; 8 (1): 2215.
3. Onikubo et al. Cell Rep. 2015. doi: 10.1016/j.celrep.2015.02.038. 25772360 PubMed PMID.
4. Lu and Wang. Frontiers in oncology. 2017; 7:241.
5. Brunetti et al. International journal of hematology. 2019.
6. Hemmati et al. Frontiers in oncology. 2017; 7:265.
7. Luger et al. Nature. 1997; 389 (6648): 251-60.
8. Simpson. Biochemistry. 1978; 17 (25): 5524-31.
9. Zhou and Bai. Essays in biochemistry. 2019; 63 (1): 75-87.
10. Di Liegro et al. Genes. 2018; 9 (6).
11. Fyodorov et al. Nat Rev Mol Cell Biol. 2018; 19 (3): 192-206.
12. De Braekeleer et al. Future oncology (London, England). 2014; 10 (3): 475-95.
13. Dovey et al. Blood. 2017; 130 (17): 1911-22.
14. Uckelmann and Armstrong. Stem Cell Reports. 2020; 15 (1): 6-12.
15. Kühn et al. Cancer discovery. 2016; 6 (10): 1166-81.
16. Deshpande et al. Blood. 2013; 121 (13): 2533-41.
17. Grummitt et al. J Biol Chem. 2008; 283 (34): 23326-32.
18. Swaminathan et al. Mol Cell Biol. 2005; 25 (17): 7534-45.
19. Gadad et al. Biochemistry. 2011; 50 (14): 2780-9.
20. Warren and Shechter. J Mol Biol. 2017; 429 (16): 2401-26.
21. Warren et al. Nat Commun. 2017; 8 (1): 2215. Epub 2017 Dec. 22.
22. Senapati et al. Methods Mol Biol. 2015; 1288:375-94.
23. Okuwaki et al. Nucleic Acids Res. 2012; 40 (11): 4861-78.
24. Shandilya et al. Mol Cell Biol. 2009; 29 (18): 5115-27.
25. Chick et al. Nat Biotech. 2015; 33 (7): 743-9.
26. Miller and Heald. J Cell Bio. 2015; 209 (2): 211-20.
27. Kashiwaya et al. Cancer Res. 2010; 70 (10): 4024-33.
28. Janke et al. EMBO Rep. 2008; 9 (7): 636-41.
29. van Dijk et al. J Biol Chem. 2008; 283 (7): 3915-22.
30. Regnard et al. J Biol Chem. 2000; 275 (21): 15969-76.
31. Ye et al. Nat Commun. 2018; 9 (1): 1261.
32. Pathak et al. Mol Biol Cell. 2014; 25 (12): 1836-44.
33. Berezniuk et al. J Biol Chem. 2013; 288 (42): 30445-53. Epub 2013 Sep. 12. doi: 10.1074/jbc.M113.497917.
34. Rogowski et al. Cell. 2010; 143 (4): 564-78.
35. Brunetti et al. Cancer Cell. 2018; 34 (3): 499-512.e9.
36. Mahalingan et al. Nat Struct Mol Biol. 2020; 27 (9): 802-13.
37. Evans et al. bioRxiv. 2022:2021.10.04.463034.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web and/or the National Center for Biotechnology Information (NCBI) on the World Wide Web.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

What is claimed:

1. A method of reducing or eliminating cellular proliferation of a cell, the method comprising contacting the cell with a composition comprising an inhibitor of Tubulin-Tyrosine Ligase Like 4 (TTLL4), wherein the cell comprises an Nucleophosmin (NPM1) protein.

2. A method of reducing or eliminating glutamate-glutamylation of NPM1 in a cell, the method comprising contacting the cell with a composition comprising an inhibitor of TTLL4.

3. The method of claim 1 or 2, wherein the inhibitor of TTLL4 is an inhibitory nucleic acid, a small molecule inhibitor, or an antibody or fragment thereof, that specifically binds TTLL4 or a nucleic acid molecule encoding TTLL4.

4. The method of claim 3, wherein the inhibitor is a small molecule.

5. The method of claim 4, wherein the small molecule specifically binds to TTLL4.

6. The method of claim 4 or 5, wherein the small molecule interacts with amino acid residue F666, 1719, K721, R727, G728, Q749, R750, Y751, L752, K762, D764, R766, R788, H807, L808, T809, N810, Y811, S812, K815, K833, D893, E906, M895, L905, E906, N908, 1909, S912, H914, D920, and/or K924 of TTLL4.

7. The method of claim 1 or 2, wherein the small molecule inhibitor interacts with amino acid residue F666 and/or 1909.

8. The method of claim 3, wherein the inhibitory nucleic acid molecule is an siRNA, miRNA, or shRNA.

9. The method of claim 8, wherein the inhibitory nucleic acid molecule is at least at least 80%, 85%, 90%, 95%, or 100% complementary to the nucleic acid sequence encoding the TTLL4.

10. The method of claim 7 or 8, wherein the inhibitory nucleic acid molecule comprises at least one modified nucleotide.

11. The method of any one of claims 7-10, wherein the composition further comprises a vector comprising a nucleic acid sequence encoding the inhibitory nucleic acid molecule.

12. The method of claim 11, wherein the vector is an expression vector.

13. The method of claim 11 or 12, wherein the vector is a viral vector.

14. The method of any one of claims 1-13 further comprising detecting the glutamate-glutamylation levels of NPM1c prior to contacting the cell, after contacting the cell, or both prior and after contacting the cell.

15. A method of reducing or eliminating cellular proliferation of a cell, or inducing differentiation of a cell, the method comprising contacting the cell with a guide RNA (gRNA) that is at least 80%, 85%, 90%, 95%, or 100% complementary to a nucleic acid sequence in the TTLL4 gene and one or more components of a CRISPR/Cas system or a nucleic acid molecule encoding the one or more components of a CRISPR/Cas system, wherein the cell comprises NPM1.

16. A method of reducing or eliminating glutamate-glutamylation of NPM1 in a cell, the method comprising contacting the cell with a guide RNA (gRNA) that is at least 80%, 85%, 90%, 95%, or 100% complementary to a nucleic acid sequence in the TTLL4 gene and one or more components of a CRISPR/Cas system or a nucleic acid molecule encoding the one or more components of a CRISPR/Cas system.

17. A method of modifying the TTLL4 gene in a cell, the method comprising contacting the cell with a guide RNA (gRNA) that is at least 80%, 85%, 90%, 95%, or 100% complementary to a nucleic acid sequence in the TTLL4 gene and one or more components of a CRISPR/Cas system or a nucleic acid molecule encoding the one or more components of a CRISPR/Cas system, wherein the cell comprises NPM1.

18. The method of any one of claims 15-17, wherein the gRNA comprises at least one modified nucleotide.

19. The method of any one of claims 15-17, wherein the nuclease is a Cas9 nuclease.

20. The method of claim 19, wherein the nuclease is a Cas9 nickase.

21. The method of claim 19, wherein the nuclease is a Cas9 cleavase.

22. The method of claim 19, wherein the Cas9 nuclease introduces a double-stranded break in the TTLL4 gene, thereby reducing or silencing expression of the TTLL4 gene.

23. The method of any one of claims 1-22, wherein the cell is a hematopoietic/progenitor stem cell.

24. The method of any one of claims 1-20, wherein the cell is an acute myeloid leukemic cell.

25. The method of any one of claims 1-24, wherein the contacting is in vitro or in vivo.

26. A cell made by the method of any one of claims 1-25.

27. A method for treating a cancer in a subject, the method comprising administering to the subject a composition comprising an inhibitor of TTLL4.

28. The method of claim 27, wherein the inhibitor is an inhibitory nucleic acid, a small molecule inhibitor, or an antibody, or fragment thereof, that specifically binds TTLL4 or a nucleic acid molecule encoding TTLL4.

29. The method of claim 27 or 28, wherein the inhibitor is a small molecule.

30. The method of claim 29, wherein the small molecule specifically binds to TTLL4.

31. The method of claim 29 or 30, wherein the small molecule interacts with amino acid residue F666, 1719, K721, R727, G728, Q749, R750, Y751, L752, K762, D764, R766, R788, H807, L808, T809, N810, Y811, S812, K815, K833, D893, E906, M895, L905, E906, N908, 1909, S912, H914, D920, and/or K924 of TTLL4.

32. The method of claim 31, wherein the inhibitor of TTLL4 interacts with F666 and/or I909.

33. The method of claim 28, wherein the inhibitory nucleic acid molecule is an siRNA, miRNA, or shRNA.

34. The method of claim 33, wherein the inhibitory nucleic acid molecule is at least at least 80%, 85%, 90%, 95%, or 100% complementary to the nucleic acid sequence encoding the TTLL4 in a cell.

35. The method of claim 33 or 34, wherein the inhibitory nucleic acid molecule comprises at least one modified nucleotide.

36. The method of any one of claims 33-35, wherein the composition further comprises a vector comprising a nucleic acid sequence encoding the inhibitory nucleic acid molecule.

37. The method of claim 36, wherein the vector is an expression vector.

38. The method of claim 36 or 37, wherein the vector is a viral vector.

39. The method of any one of claims 27-38, further comprising detecting the glutamate-glutamylation levels of NPM1 prior to contacting the cell, after contacting the cell, or both prior and after contacting the cell.

40. A method of treating a cancer in a subject, the method comprising administering to the subject a guide RNA (gRNA) that is at least 80%, 85%, 90%, 95%, or 100% complementary to a nucleic acid sequence in the TTLL4 gene and one or more components of a CRISPR/Cas system or a nucleic acid molecule encoding the one or more components of a CRISPR/Cas system.

41. The method of claim 40, wherein the gRNA comprises at least one modified nucleotide.

42. The method of claim 40 or 41, wherein the nuclease is a Cas9 nuclease.

43. The method of claim 42, wherein the nuclease is a Cas9 cleavase.

44. The method of claim 42, wherein the nuclease is a Cas9 nickase.

45. The method of claim 42 or 43, wherein the Cas9 nuclease introduces a double-stranded break in the TTLL4 gene in a cell, thereby reducing or silencing expression of the TTLL4 gene in the cell.

46. The method of claim 34 or 45, wherein the cell is a hematopoietic/progenitor stem cell.

47. The method of claim 34 or 45, wherein the cell is an acute myeloid leukemic cell.

48. The method of any one of claims 27-47, wherein the gRNA and the CRISPR/Cas system are coadministered.

49. The method of any one of claims 27-47, wherein the gRNA and the CRISPR/Cas system are present in a pharmaceutical composition further comprising a pharmaceutically acceptable carrier.

50. The method of any one of claims 27-48, wherein the gRNA is present in a first pharmaceutical composition further comprising a pharmaceutically acceptable carrier and the CRISPR/Cas system is present in a second pharmaceutical composition further comprising a pharmaceutically acceptable carrier.

51. The method of any one of claims 27-47, wherein the gRNA and the CRISPR/Cas system are administered sequentially.

52. The method of any one of claims 27-51, further comprising detecting the level of TTLL4 protein or polynucleotide and/or NPM1 glutamate-glutamylation.

53. The method of claim 52, wherein the detecting is performed prior to administration or after administration.

54. The method of claim 52, wherein the detecting is performed prior to and after administration, wherein a decrease in the level of TTLL4 and/or NPM1 glutamate-glutamylation is indicative of therapeutic effectiveness.

55. The method of any one of claims 1-54 wherein the NPM1 is NPM1c.

56. The method of any one of claim 3 to 7 or 28-32, wherein the small molecule inhibitor has the structure of Formula (I): 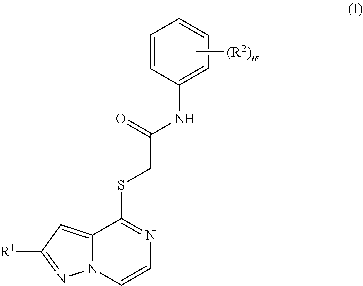
or a pharmaceutically acceptable salt thereof, wherein:
R¹ is (C₁-C₁₀) aryl, which is optionally substituted with one to three substitutents selected from (C₁-C₆)alkyl, (C₁-C₆) fluoroalkyl, (C₁-C₆)alkoxy and chloro;
R² is independently for each occurrence (C₁-C₆)alkyl, (C₁-C₆)alkoxy, (C₁-C₆)thioalkoxy, acetyl, cyano, fluoro, and chloro; and
n is 0, 1, 2, or 3.

57. The method of claim 56, wherein R¹ is phenyl optionally substituted with one to three substitutents selected from (C₁-C₆)alkyl, (C₁-C₆)alkoxy and chloro.

58. The method of claim 57, wherein R¹ is phenyl, 4-methylphenyl, 4-chlorophenyl, 4-ehtylphenyl, 4-methoxyphenyl, 2-methoxyphenyl, 4-butoxyphenyl, or napthalen-2-yl.

59. The method of anyone of claims 56 to 58, wherein R² is independently for each occurrence selected from methyl, ethyl, isopropyl, chloro, fluoro, cyano, methoxy, acetyl, thiomethoxy, and trifluoromethyl.

60. The method of any one of claims 56 to 59, wherein nis 1.

61. The method of any one of claims 56 to 59, wherein n is 2.

62. The method of any one of claims 56 to 59, wherein nis 3.

63. The method of any one of claims 56 to 59, wherein: 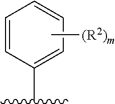
is:
phenyl, 4-methylphenyl, 2,4,6-trifluoromethylphenyl, 4-ethylphenyl, 2-methylphenyl, 2,6-dimethylphenyl, 2,5-dimethylphenyl, 2,4-dimethylphenyl, 4-isopropyl, 3,4-dimethylphenyl, 3-chlorophenyl, 2-chlorophenyl, 4-chlorophenyl, 2-chloro-4-methylphenyl, 2-methyl-4-chlorophenyl, 2-methyl-3-chlorophenyl, 3-fluorophenyl, 4-fluorophenyl, 2,4-difluorophenyl, 2-methyl-5-fluorophenyl, 4-methyl-5-fluorophenyl, 2,5-difluorophenyl, 3-methoxyphenyl, 2-methoxyphenyl, 4-methoxyphenyl, 2,4-dimethoxyphenyl, 2,5-dimethoxyphenyl, 3,4-dimethoxyphenyl, 3-thiomethoxyphenyl, 2-trifluormethylphenyl, 4-acetylphenyl, 2-trifluoromethyl-4-chlorophenyl, and 3-cyanophenyl.

64. The method of any one of claims 3 to 7 or 28-32, wherein the small molecule inhibitor has the structure of Formula (II): 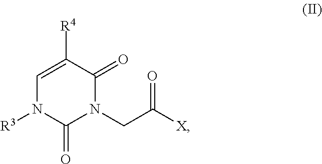
or a pharmaceutically acceptable salt thereof, wherein:
R³ is (C₃-C₈) cycloalkyl, 4- to 7-membered hetercyloalkyl, and 5- to 6-membered heteraryl;
R⁴ is hydrogen, fluoro, chloro, bromo, (C₁-C₆)alkyl, or (C₁-C₆) haloalkyl;
X is 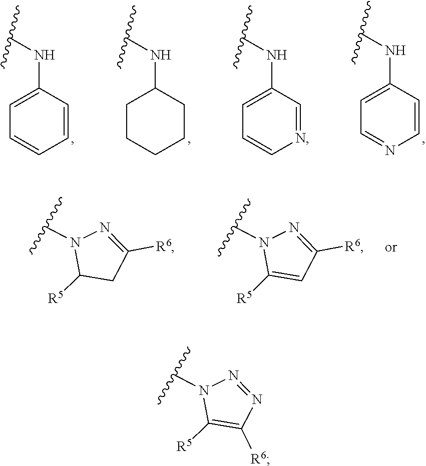
and
R⁵ and R⁶ are each independently hydrogen, furanyl, thiophenyl, or phenyl, wherein phenyl is optionally substituted with one, two or three substituents selected independently from fluoro, chloro, bromo, hydroxy, (C₁-C₆)alkoxy, and NH₂.

65. The method of claim 64, wherein R³ is cyclopentyl, tertrahydrofuran-3-yl, tetrahydrothiophen-3-yl, tetrahydrothiophen-2-yl, furan-2-yl, pyrrolidine-2-yl, or pyrrol-2-yl.

66. The method of claim 64 or 65, wherein R⁴ is fluoro.

67. The method of any one of claims 64 to 66, wherein R⁵ is thiophen-2-yl, furan-2-yl, phenyl, 4-chlorophenyl, or hydrogen.

68. The method of any one of claims 64 to 67, wherein R⁶ is 4-chlorphenyl, 4-fluorophenyl, 4-bromophenyl, 4-hydroxyphenyl, 4-aminophenyl, phenyl, 4-methoxyphenyl or hydrogen.

69. The method of any one of claims 64 to 68, wherein X is:

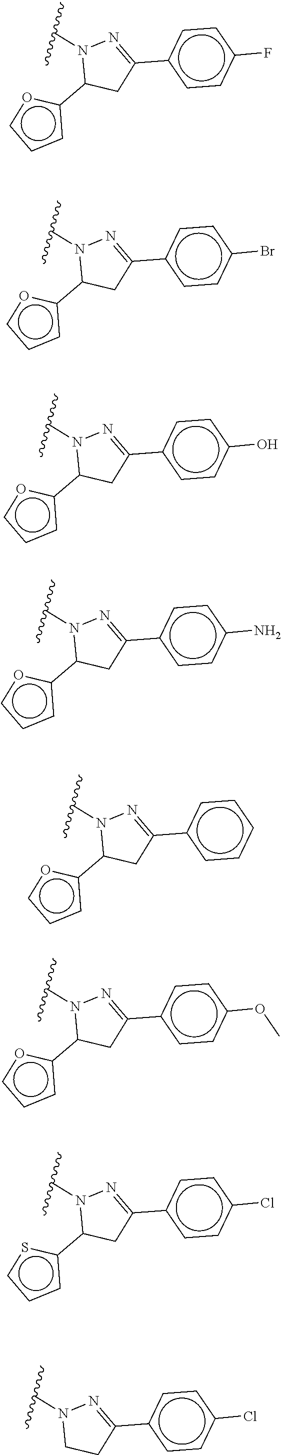

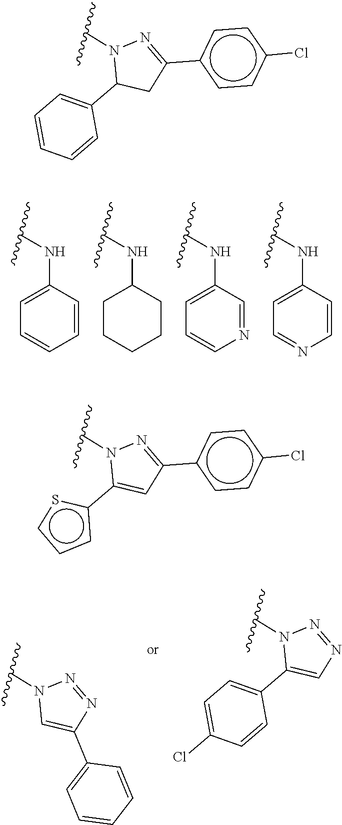

70. The method of any one of claim 3 to 7 or 28-32, wherein the small molecule inhibitor is: 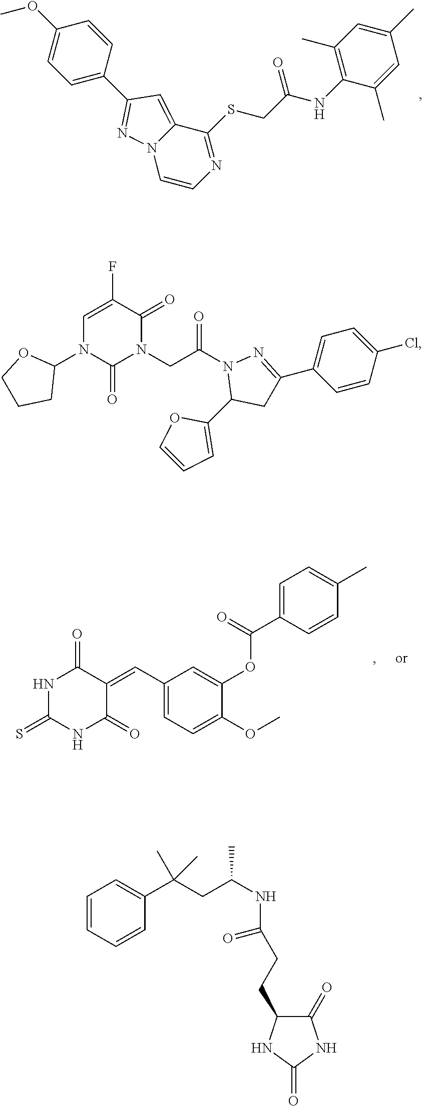
or a pharmaceutically acceptable salt thereof.

71. The method of any one of claims 28 to 55, wherein the cancer is associated with overexpression of TTLL4.

72. The method of claim 71, wherein the cancer is renal cell kidney cancer, melanoma, or glioblastima multiforme.

73. The method of any one of claims 28 to 55, wherein the cancer is acute myeloid lymphoma.
